(19) United States
(12) Patent Application Publication
(10) Pub. No.: US 2006/0177356 A1
(43) Pub. Date: Aug. 10, 2006

(54) Positive pressure air purification and conditioning system

(76) Inventor: Gregory Robert Miller, Washington, MO (US)

Correspondence Address: HUSCH & EPPENBERGER, LLC, 190 CARONDELET PLAZA, SUITE 600, ST. LOUIS, MO, 63105-3441, US

(21) Appl. No.: 11/054,521

(22) Filed: Feb. 8, 2005

(51) Int. Cl. A61L 9/20

(52) U.S. Cl. 422/121

(57) ABSTRACT

An air purification system utilizing an ultraviolet germicidal lamp is disclosed. The air purification system is in fluid communication with a ventilation duct. The air purification system may also be in fluid communication with the ambient air. The air purification system may incorporate an actuator for the selection of the ambient air, the ventilation duct, or both as an air source for treatment. The air purification system may also be used to obtain a positive pressure within an enclosed space.

### CROSS-REFERENCE TO RELATED APPLICATIONS

Not applicable.

### BACKGROUND OF THE INVENTION

1. Field of the Invention

The present invention relates generally to air cleansing devices and, more particularly, to ultraviolet irradiation and filtration devices.

2. Related Art

Over the years, many devices have been used to treat the air in an attempt to purify it. A common strategy is to use an air filter to rid indoor air of biological contaminants. While this is an important element of cleaning air, this has its problems. Most filters are inadequate because many organisms pass right on through the filter due to the limitations of filter size. Also, any organisms that collect on the filter can form germ colonies that may soon contaminate passing air. Further, if the filter should be too efficient, it blocks the passage of air and creates back pressure, causing the blower to struggle to move air through the system. Moreover, even if air within a room has been treated and purified, the room is once again contaminated once the HVAC system begins operation. In other words, the HVAC system deposits biological contaminants via a ventilation duct and “stirs up” contaminants that may have settled within the room.

Another strategy to treat air is the use of an ultraviolet lamp. Ultraviolet (UV) light in the form of germicidal lamps has been used since the early 1900's to kill the same types of microorganisms that typically cause the same types of problems today. UV radiation in the short wave or C-band range (UVC) is used in a wide range of germicidal applications to destroy bacteria, mold, yeast and viruses. Typical applications included hospitals, beverage production, meat storage and processing plants, bakeries, breweries, pharmaceutical production and animal laboratories; virtually anywhere microbial contamination is of concern.

The ability of ultraviolet light to decompose organic molecules has been known for a long time. Most organic molecules have a strong absorption band between 200 nm and 300 nm. In comparison, UVC is generally understood to exist in the 180 nm to 280 nm wave length area. A wave length of 253.7 nm is useful for exciting and disassociating contaminant molecules, but 265 nm is thought to be the optimal spectral line for germicidal effectiveness.

There remains a need in the art for a device for the treatment of air that avoids the problems presented by the engagement and operation of an HVAC system. Further, there remains a need in the art for a device and method to apply ultraviolet germicidal irradiation to an HVAC air stream.

### SUMMARY OF THE INVENTION

The invention is an air purification unit for the achievement of better indoor air quality. The air purification unit draws air in, treats the drawn air with an ultraviolet germicidal lamp to remove contaminants, and blows the treated air into a room or office. Generally, the air purification unit includes a housing having an inlet and an outlet, an air treatment chamber, an ultraviolet lamp located within the air treatment chamber, and an air handler for moving air from the inlet to the outlet. The air purification unit may be configured in different ways. As examples, the air purification unit may take the form of a “floor hugger” model or a “tower” model. The air purification unit may draw air in from an ambient air source, a ventilation duct, or some combination thereof. Therefore, the air purification system may be used to generally improve indoor air quality, or it may be used to purify an HVAC air stream.

The invention described herein purifies air, either ambient air or air received from a ventilation duct. When the unit is installed in a room and the room is equipped with a return air duct, it is possible, depending on the air flow and air purification capability of the room unit, to distribute increased purity throughout the rest of the home. The unit, as an example, running more or less continuously and drawing air from the HVAC supply duct, will cause air to be drawn into all open return air ducts and said air will return ultimately to the room air purification unit from the supply ducts.

One embodiment of the air purification system has an upper (or first) portion and a lower (or second) portion, each with its own air handler. The use of twin air handlers is significant because two air handlers operating at 50 percent capacity are quieter than a single air handler operating at 100 percent capacity. Each portion is connected to an air source. As an example, the upper or first portion may be connected to an ambient air source and the lower or second portion may be connected to a ventilation duct. Further, the air handlers may operate independently of one another. Therefore, a user may adjust the speed of an air handler to draw more or less air from a particular source. Additionally, the lower portion air handler may turn “on” and “off” in response to an airflow or air temperature or other sensor means when the lower ventilation duct supplies forced air flow. Further, the UV lamp and/or other air purification means in the upper or lower portion may be activated on or off in response to air flow, temperature, etc. within said lower or upper portion of the purification system.

The air purification unit is particularly useful in maintaining a positive pressure system or environment. A positive pressure system reduces the overall air purification requirements of a room by blocking typical sources of indoor air contamination. Typically, air flows into an enclosed space bringing with it additional contaminants. By creating a positive pressure, air flows out of the enclosed space, thereby preventing entry of the additional contaminants. By using the air purification unit to bring air in from outside the room while contemporaneously forcing air within the room to exit via predetermined path, a positive pressure is achieved. The combination of purifying air brought into the room and the positive pressure to prevent further contamination leads to exceptional room air quality.

The air purification unit is adapted to receive air from more than one source. As such, some embodiments of the air purification unit include controls to select a source of air for purification. For example, the air purification unit may receive both room air and air from an HVAC duct. The air purification unit may select the room air, air from the HVAC air stream, or some combination thereof for filtration. Source selection may be accomplished in at least two different ways. First, the air purification unit may control the amount of air drawn in from one of the inlets. This may be accomplished through the use of a grill or a damper. Second, the air purification system may adjust the speed of the air handler to draw in more or less air.

The air purification unit may also be used to regulate a temperature within a room. The air purification may have a heater or may receive conditioned air from another source. For example, the air purification unit may receive heated or cooled air from an HVAC duct. The air purification unit may use its own heater or the conditioned air to maintain a specified room temperature.

Some embodiments of the air purification unit are equipped with a compressor. The compressor can be used to route treated air to virtually any type of personal breathing device, such as masks, cannulas, nebulizers, or the like. In this manner, the air purification unit can be used to supply a purified air stream to personal breathing devices which may otherwise be subjected to unpurified air.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a schematic of an air purifier system;

FIG. 2 is a perspective view of the air purifier system in a first embodiment;

FIG. 3 is a sectional top view of the air purifier system;

FIG. 4 is a sectional side view of a second embodiment of the air purifier system;

FIG. 5 is a schematic of the air purifier system;

FIG. 6 is a sectional side view of the third embodiment of the air purifier system;

FIG. 7 is a sectional top view of the third embodiment of the air purifier system;

FIG. 8 is a sectional top view of the air purifier system having electrically charged plates;

FIG. 9 is a sectional side view of the fourth embodiment of the air purifier system;

FIG. 10 is a sectional top view of the fourth embodiment of the air purifier system;

FIG. 11 is a sectional top view of the fourth embodiment of the air purifier system;

FIG. 12 is a perspective view of a fifth embodiment of the air purifier system;

FIG. 13 is a side view of the sixth embodiment of the air purifier system in a first configuration;

FIG. 14 is a side view of the sixth embodiment of the air purifier system in a second configuration;

FIG. 15 is a sectional side view of the sixth embodiment of the air purifier system in a third configuration;

FIG. 16 is a sectional side view of the sixth embodiment of the air purifier system in a fourth configuration;

FIG. 17 is a perspective view of a positive pressure system;

FIG. 18 is a flow chart illustrating a control system for the positive pressure system;

FIG. 19 is a perspective view of a source selection switch;

FIG. 20 is a second embodiment of the source selection switch;

FIG. 21 is a schematic of the source selection system;

FIG. 22 is a perspective view of a third embodiment of the source selection switch;

FIG. 23 is a sectional side view of the embodiment shown in FIG. 24;

FIG. 24 is a sectional side view of the source selection control system;

FIG. 25 is a schematic of the source selection control system;

FIG. 26 is a front view of a nose mask;

FIG. 27 is a front view of a cannula in a first embodiment;

FIG. 27A is a front view of a cannula of the first embodiment of FIG. 27, but with the added feature of removable attachment;

FIG. 28 is an elevated view of a cannula in a second embodiment;

FIG. 28A is an elevated view of the cannula of FIG. 28, but with the alternative feature of prioritized nasal oxygen flow, and inspiratory effort sensing;

FIG. 29 is a front view of a cannula in a third embodiment;

FIG. 30 is a perspective view of a face mask;

FIG. 31 is a front view of a nebulizer;

FIG. 32 is a front view of cooling coil;

FIG. 33 is a front view of a water trap;

FIG. 34 is a front view of an oxygen bottle;

FIG. 35 is a flow chart illustrating operation of the air purification system;

FIG. 36a is a flow chart illustrating maintenance of room temperature utilizing the air purification system;

FIG. 36b is a continuation of the flow chart shown in FIG. 36a;

FIG. 36c is a continuation of the flow chart shown in FIG. 36a;

FIG. 37 is a flow chart illustrating a setup operation of the air purification system; and

FIG. 38 is a flow chart illustrating maintaining a room temperature utilizing the air purification system.

### DETAILED DESCRIPTION OF THE PREFERRED EMBODIMENTS

The following description of the preferred embodiment(s) is merely exemplary in nature and is in no way intended to limit the invention, its application, or uses.

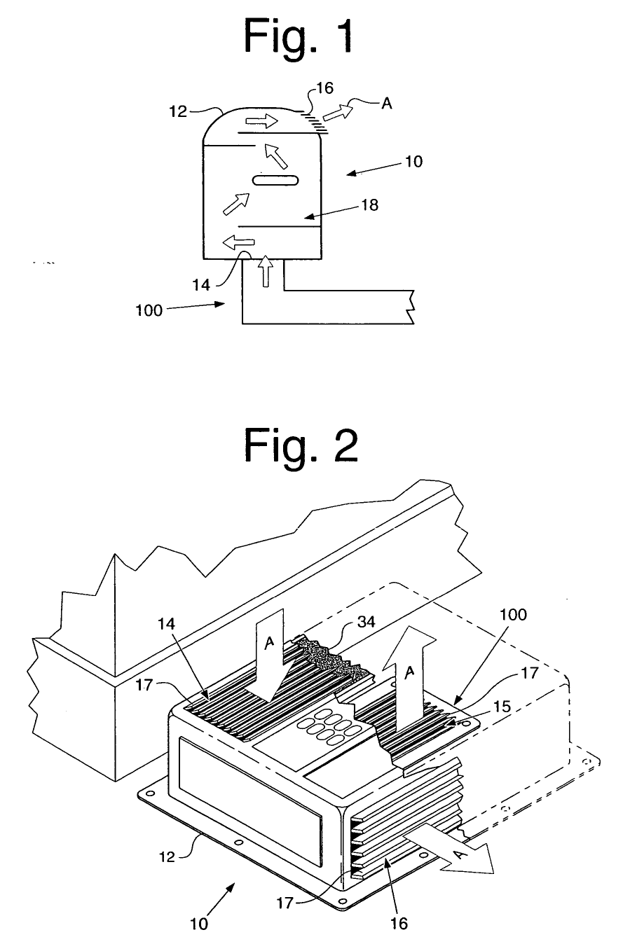

Referring to the accompanying drawings in which like reference numbers indicate like elements, FIG. 1 illustrates an air purification system 10. The air purification system 10 is used to create and sustain exceptionally high indoor air quality in a room or a series of rooms. The air purification system 10 utilizes a ventilation duct 100, such as Heating, Ventilation, and Air Conditioning (HVAC) supply and return ducts, which are commonly found in many houses and buildings. Further, the air purification system 10 may be used in a room equipped with a closable door or doors. While the preferred embodiments of the air purification system 10 are discussed in relation to a bedroom in a home, those skilled in the art would understand that the air purification system 10 could also be used in an office in a building, a group of offices, or other similar locations.

Base Unit

The air purification system 10 includes a housing 12. The housing 12 may be configured in numerous ways. In the embodiment depicted in FIG. 1, the housing 12 is configured as a “floor hugger.” The housing 12 includes at least one inlet 14, a treatment chamber 18, and at least one outlet 16. The air A enters the air purification system 10 through the inlet 14, the air A is treated in the treatment chamber 18, and then the air A exits through the outlet 16. In the depicted embodiment, the inlet 14 is in fluid communication with the ventilation duct 100. As an example, heated air from an HVAC system (not shown) may travel through the ventilation duct 100 and into the air purification system 10 through the inlet 14.

FIG. 2 illustrates an alternative embodiment of the air purification system 10 having two inlets. In the embodiment depicted in FIG. 2, the housing 12 includes a first inlet 14, a second inlet 15, and an outlet 16. Air A enters the first inlet 14 and the second inlet 15, the air A is treated within the housing 12, and the air A exits through the outlet 16.

As examples, the first inlet 14 may be connected to ambient air and the second inlet 15 may be aligned with the ventilation duct 100 to receive air from an HVAC unit. In some embodiments, the air purification system 10 may include a nylon or felt seal in-between the second inlet 15 and the ventilation duct 100. Because the air purification system has more than one inlet, the air purification system 10 may receive unpurified air from more than one source. Further, the air purification system 10 may include controls to select an air source. For example, a user may select to purifier air received by the first inlet 14, the second inlet 15, or some combination thereof. Air source selection will be explained in greater detail below.

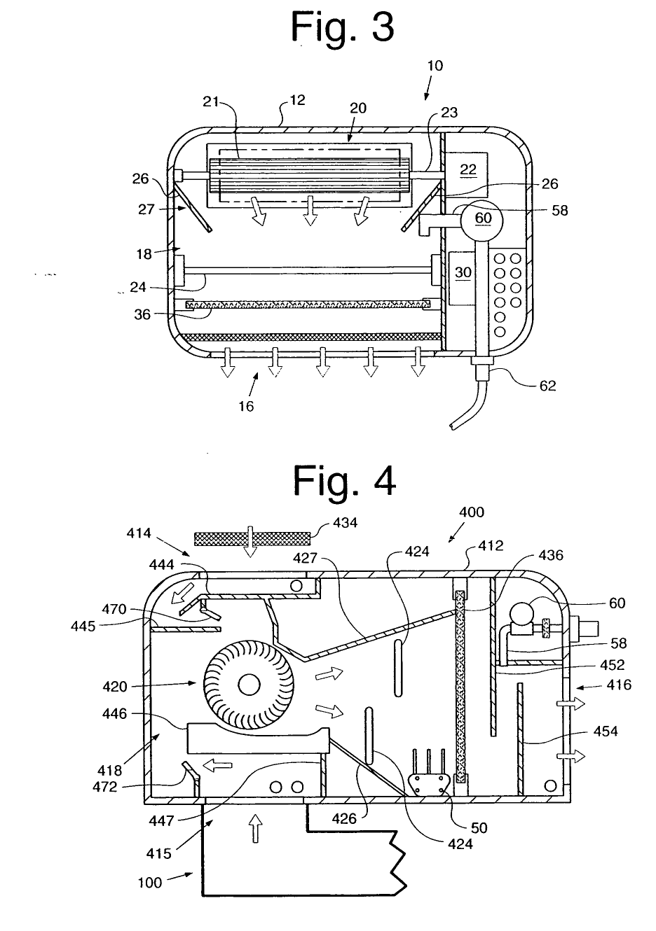

FIG. 3 illustrates one example of the air treatment chamber 18. In the depicted embodiment, the treatment chamber 18 includes an air handler 20. It is preferred that the air handler be within direct or reflected UV light to remain sterile and enhance self-cleaning through decomposition of any collected organic material. Aluminum is a preferred tangential blower material, although other non-organic materials will work as well. The principal function of the air handler 20 is to move air into and out of the air purification system 10. The air handler 20 has a bearing (not shown) mounted at each end and supported by the housing 12. The air handler 20 also includes a motor 22. The motor 22 is connected to a shaft 23, which is connected to fan blades 21. Thus, the motor 22 rotates the fans blades 21 via the shaft 23 for air movement. In the depicted embodiment, the air handler 20 is a tangential blower provided by Eucania International, Inc, with offices at 275 Guthrie Avenue, Dorval, Quebec, Canada. In some embodiments, the motor-end of the air handler 20 may require additional structure and mass if the air handler 20 lacks a bearing and shaft support on the non-motor end.

The treatment chamber 18 also includes a UV light 24. In the embodiment depicted in FIG. 3, there is one UV light 24, but those skilled in the art would understand that a greater number of lights may be used. As an example only, the UV light 24 may be a cold cathode germicidal ultraviolet lamp as provided by Atlantic Ultraviolet Corporation, which has an office at 375 Marcus Boulevard, Hauppauge, N.Y., U.S.A. The term “UV light” should be understood within the body of invention to mean any source of man made UV light technology including lamps, LEDs, etc. The compactness of the design of the air treatment chamber 18 ensures that all of the air that passes through the air purification system 10 comes within close proximity to the light 24. The term “close proximity” means the air is subjected to maximum ultraviolet germicidal intensity to kill, sterilize and disintegrate airborne contaminants.

The light 24 is connected to a power supply 30. In the depicted embodiment, the power supply 30 in an indoor power supply provided by Ventex Technology, Inc., with offices at 7830 Byron Drive #10, Riviera Beach, Fla., U.S.A. The power supply 30 is used to supply the appropriate amount of current to the UV light 24.

The air handler 20 may be constructed to allow the insertion of the UV lamp 24 into its center. In that case, a shaft and bearing of proper size can be constructed on the non-motor end such that the shaft could be hollow to allow wiring to pass through it. A flange to support the UV lamp 24 could be attached to the shaft and a bearing secured to a removable flange at the end of the air handler 20 would be required for assembly, disassembly, and lamp replacement.

The treatment chamber 18 further includes air guides 26. The air guides 26 are used in conjunction with the air handler 20 to direct the air towards the light 24. The air guides 26 may be a simple plastic or metal plate mounted to the housing 12. In some embodiments, the lamp-side of the air guides 26 may have a reflective coating 27 to reflect light provided by the UV light 24. Light reflected from the reflective coating 27 will aid in treating the air A within the treatment chamber 18.

Additionally, the air purification system 10 may utilize a self-cleaning filter mesh 36 downstream of the light 24 to collect positively-charged particulates for destruction and disintegration. In the depicted embodiments, the filter mesh 36 is made of aluminum but other materials may be used. The filter mesh 36 is easily removable for inspection and cleaning, if necessary.

Referring once again to FIG. 2, the first inlet 14, the second inlet 15, and the outlet 16 each have a louvered cover 17 to keep fingers out of the air purification system 10. This will prevent a user from coming into contact with the air handler 20. The louvered cover 17 will also prevent direct line of sight contact into the air purification system 10 as light from the UV lamp 24 may be detrimental. In some embodiments, the louvers of the louvered cover 17 are pivotable, either manually or automatically. As such, it may possible to adjust the opening of the first inlet 14 or the second inlet 15. As an example, the louvered cover 17 may be a zone damper available from Jackson Systems, LLC, 100 East Thompson Road, Indianapolis, Ind., U.S.A. It is also contemplated that the louvered cover 17 may oscillate to push air over a broader circulation path.

Further, in some embodiments the air purification system 10 is equipped with a removable air inlet filter 34. The air inlet filter 34 may be used in instances when the end user desires to use the air purification system in a configuration that does not extract filtered air from the ventilation duct 100. It is anticipated, but not necessary, that the air coming from the ventilation duct 100 will have undergone some sort of gross particulate collection. The use of the air inlet filter 34 is at the user's discretion. An air inlet filter may also be used between the ventilation duct and the air purification system at that user's discretion.

FIG. 4 illustrates an alternative embodiment of the air purification system, generally indicated by numeral reference 400. In the embodiment depicted in FIG. 4, the air purification system 400 includes a housing 412, two UV lights 424, an air treatment chamber 418, a filter mesh 436, a first guide 426, a second guide 427, and an air handler 420. The air handler 420 is located within the air treatment chamber 418. In the depicted embodiment, the UV lights 424 are adjacent the air handler 420. In this manner, the air handler 420 blows the air towards the UV lights 424. The filter mesh 436 is located in between the air treatment chamber 418 and the outlet 416.

Although a single UV lamp can typically meet the requirements of most applications, the air purification system 400 includes two UV lamps 424. The two UV lamps 424 may operate simultaneously for increased purification or the UV lamps 424 may alternate in operation, thereby prolonging lamp life and extending replacement and maintenance intervals, and saving energy. The air purification system 400 includes a first inlet 414, a second inlet 415, and an outlet 416. In the depicted embodiment, the air purification system 400 is connected to the ventilation duct 100 at the second inlet 415 and receives ambient air from the first inlet 414. However, those skilled in the art will understand that the air purification system 400 may receive air solely from the first inlet 414 or the second inlet 415. The air purification system 400 includes an air inlet filter 434.

The air purification system 400 includes a first duct 444, a second duct 445, a third duct 446, and a fourth duct 447. The air purification system 400 also includes a first damper 470 and a second damper 472. As an example, the dampers 470, 472 may be electro-mechanical dampers available from Smarthome, Inc., 16542 Millikan Avenue, Irvine, Calif., U.S.A. The first damper 470 is located adjacent the first inlet 414, and the second damper 472 is located adjacent the second inlet 415. In some embodiments, the air purification system 400 may include a first baffle 452 and a second baffle 454.

Air enters the inlets 414, 415, is treated in the treatment chamber 418, and exits the outlet 416. The dampers 470, 472 open and close to regulate the air flow into the air treatment chamber 418. For example, if damper 470 is closed, then ambient air will not enter the air treatment chamber 418. Once air enters the treatment chamber 418, the air handler 420 and the ducts 444, 445, 446, 447 cooperate to move the air from the inlets 414, 415 to the outlet 416. As the air passes through the air treatment chamber 418, it is treated by the UV lamps 424.

The first guide 426 and the second guide 427 provide a passageway in which the air flows. In the embodiment depicted in FIG. 4, the first guide 426 and the second guide 427 are angled away from one another. This serves to increase the volume of the passageway as the air travels from the air handler 420 to the outlet 416. The increase in volume decreases the velocity of the air, thereby prolonging its treatment by the UV lamps 24. Additionally, some embodiments include the baffles 452, 454. The baffles 452, 454 create a slight backpressure to further slow down the air in order to prolong its treatment.

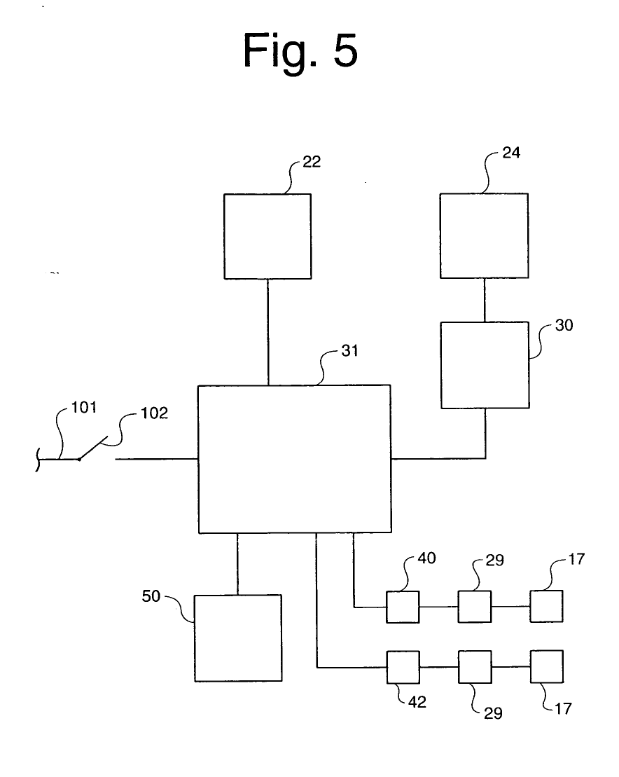

FIG. 5 illustrates a schematic of the air purification system 10, 400. The air purification system 10, 400 includes a voltage regulator 31. The voltage regulator 31 is electrically connected to a power source 101. In some embodiments, there is a switch 102 in between the power source 101 and the voltage regulator 31. The components of the air purification system 10, 400 are electrically connected to the voltage regulator 31. The motor 22 is electrically connected to the voltage regulator 31. The power supply 30 is electrically connected to the voltage regulator 31. Further, the UV lamp 24 is connected to the power supply 30. Additionally, a negative ion generator 50 may be utilized if desired, and if present, is also connected to the voltage regulator 31.

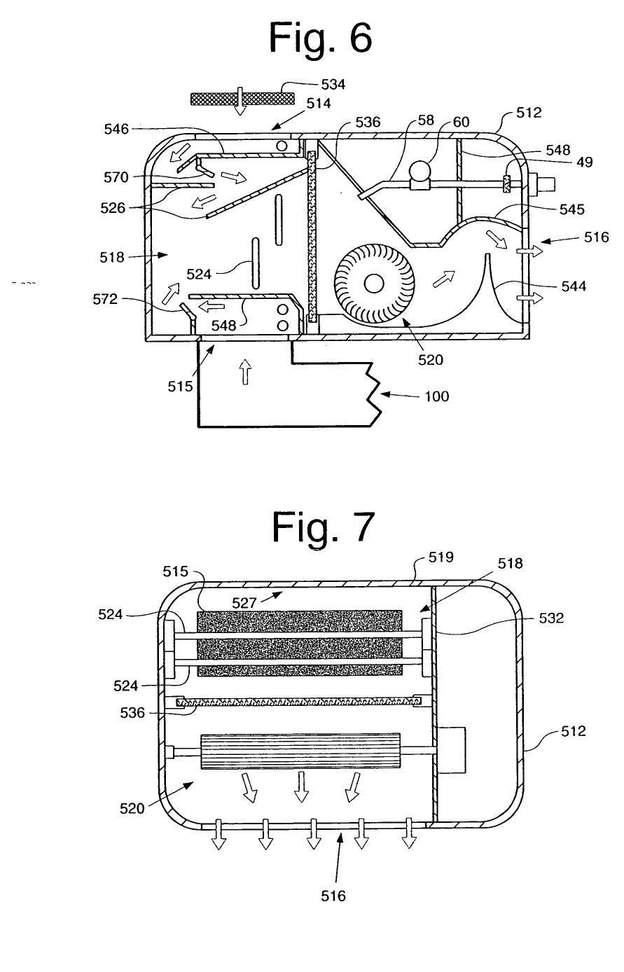

FIGS. 6 and 7 illustrate an alternative embodiment of the air purification system, generally indicated by numeral reference 500. In the embodiment depicted in FIG. 6, the air purification system 500 includes a housing 512, two UV lights 524, an air treatment chamber 518, a filter mesh 536, an air guide 526, and an air handler 520. The air handler 520 pulls the air A instead of pushing it. In the depicted embodiment, the UV lights 524 are located within the air treatment chamber 518, and the air handler 520 is located adjacent the UV lights 524. Further, the filter mesh 536 is located in-between the UV lights 524 and the air handler 520.

The air purification system 500 includes a first inlet 514, a second inlet 515, and an outlet 516. In the depicted embodiment, the air purification system 500 is connected to the ventilation duct 100 at the second inlet 515 and receives ambient air from the first inlet 514. However, those skilled in the art will understand that the air purification system 500 may receive air solely from the first inlet 514 or the second inlet 515. The air purification system 500 includes an air inlet filter 534. The air purification system 500 includes a first duct 544, a second duct 545, a third duct 546, a fourth duct 547, and a fifth duct 548. A support member 548 connects to the housing 512 and supports the second duct 545. Significantly, the first duct 544 and the second duct 545 create a tapered passageway that directs air towards the air handler 520. The tapered passageway allows the air handler 520 to pull air through the treatment chamber 518. The air purification system 500 also includes a first damper 570 and a second damper 572.

Air enters the inlets 514, 515, is treated in the treatment chamber 518, and exits the outlet 516. The dampers 570, 572 open and close to regulate the air flow into the air treatment chamber 518. For example, if damper 570 is closed, then ambient air will not enter the air treatment chamber 518. Once air enters the treatment chamber 518, the air handler 520 and the ducts 544, 545, 546, 547 cooperate to move the air from the inlets 514, 515 to the outlet 516. As the air passes through the air treatment chamber 518, it is treated by the UV lamps 524.

FIG. 7 illustrates a sectional top view of the air purification system 500. In some embodiments, a reflective material 527 is attached to a back wall 519 of the air treatment chamber 518, although it is understood that reflective material 527 may be placed on all surface walls of the air treatment chamber 518. The reflective material 527 reflects the light from the UV lamps 524 and aids in the treatment of the air in the air treatment chamber 518. The air purification system 500 includes a divider plate 532. The divider plate 532 partitions the housing 512 and provides a wall of the air treatment chamber 518.

In some embodiments, the air purification system 10, 400, 500 may include an electro-static attachment to remove unwanted dust particles and the like. For example, as best seen in FIG. 4, the negative ion generator 50 may be incorporated into the design to freshen air returning into the room. In the depicted embodiment, the negative ion generator 50 is mounted next to the outlet 16. For example, the negative ion generator 50 may be a 120 Volt Alternating Current (VAC) negative ion generator, which is available from The Electronic Goldmine, a division of Chaney Electronics, Inc., P.O. Box 5408, Scottsdale, Ariz., U.S.A. An electro-negative charge would exist via the ultraviolet germicidal irradiation and/or it could be electrically induced on the aluminum portion of the air handler 20 and/or on the filter mesh 36. The electro-negative charge would allow the capture of some positively charged particulate matter which would be easily irradiated (sterile) and broken down without imbedded or adhered chemical catalysts.

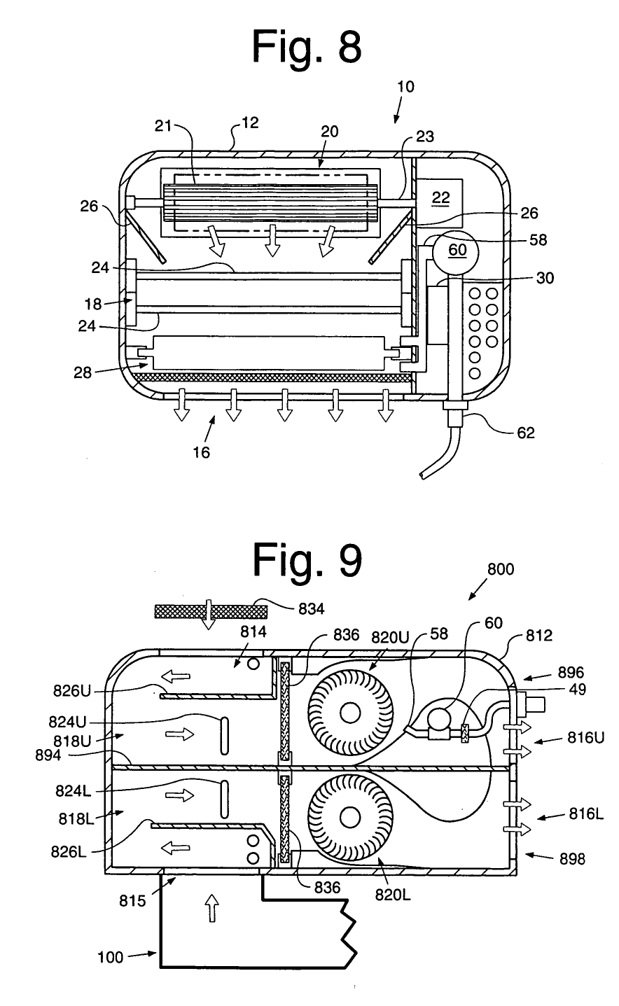

Alternatively, as best seen in FIG. 8, some embodiments may incorporate aluminum plates 28. The aluminum plates 28 may be positively or negatively charged to reduce negative ion output and to collect particulate matter. As an example, the air purification system may contain two aluminum plates 28, each having its own charge opposite of the other. The aluminum plates 28 may be removed from the housing as necessary for cleaning.

The air purification system 10 may also be equipped with a self cleaning actinism chamber filter-mesh as disclosed by U.S. Pat. No. 6,221,314, which issued to Bigelow on Apr. 24, 2001 and incorporated herein by reference in its entirety, to additionally add another layer of inorganic matter collection and disintegration.

Twin Air Handler

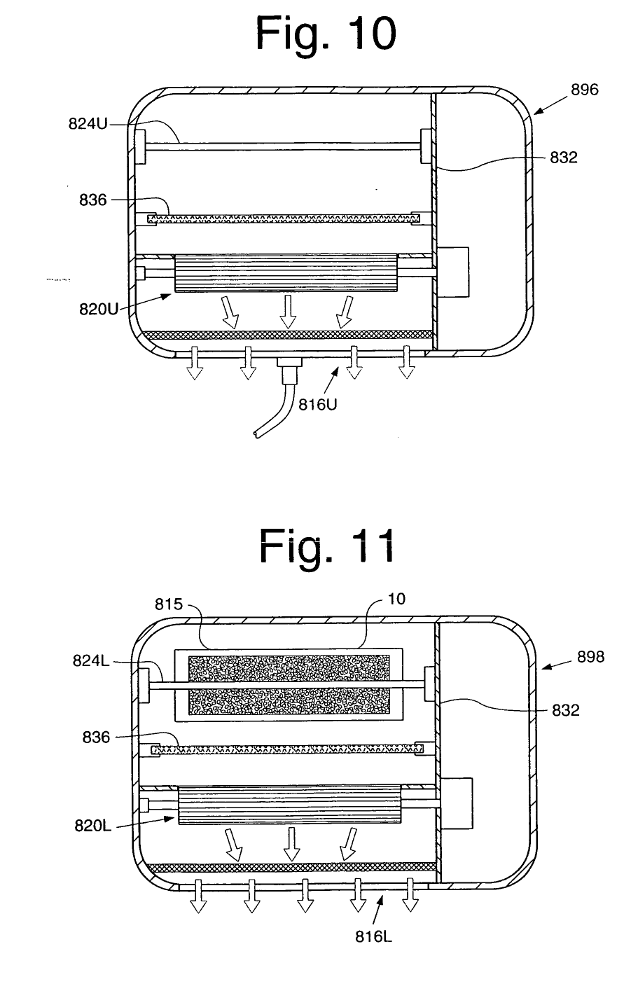

FIGS. 9, 10, and 11 illustrate an air purification system 800. The air purification system 800 includes an upper portion 896 and a lower portion 898. A partition member 894 separates the upper portion 896 from the lower portion 898. In some embodiments, the partition member 894 is made of an UV permeable material, such as quartz glass. In the embodiment depicted in FIG. 8, the air purification system 800 includes a housing 812, an upper UV light 824U, a lower UV light 824L, an upper air treatment chamber 818U, a lower air treatment chamber 818L, two filter mesh 836, an upper guide 826U, a lower guide 826L, an upper air handler 820U, and a lower air handler 820L. The air handlers 820 pull the air A instead of pushing it. Further, the use of two air handlers is significant because two air handlers operating at 50% capacity are quieter than a single air handler operating at 100% capacity. Each UV light 824U, 824L is located within the respective air treatment chamber 818U, 818L. The UV lights 824U, 824L can alternate “on” and “off” in this configuration to extend lamp life and maintenance intervals. Both UV lights 824U, 824L can be “on” when the air handlers are running at higher speeds to increase intensity. Each air handler 820U, 820L is located next to the respective UV light 824U, 824L. Further, each filter mesh 836 is located in-between the respective UV light 824U, 824L and the respective air handler 820U, 820L.

The air purification system 800 includes a first inlet 814, a second inlet 815, an upper outlet 816U, and a lower outlet 816L. In the depicted embodiment, the air purification system 800 is connected to the ventilation duct 100 at the second inlet 815 and receives ambient air from the first inlet 814. However, those skilled in the art will understand that the air purification system 800 may receive air solely from the first inlet 814 or the second inlet 815. The air purification system 800 may receive air from inlet 814 and inlet 815 when the system is positioned upright as shown in FIG. 13 and FIG. 14, and not connected to ventilation duct 100. In this alternative configuration, the air purification system is similar to standard room air purifiers. The air purification system 800 also includes an air inlet filter 834 and a divider plate 832.

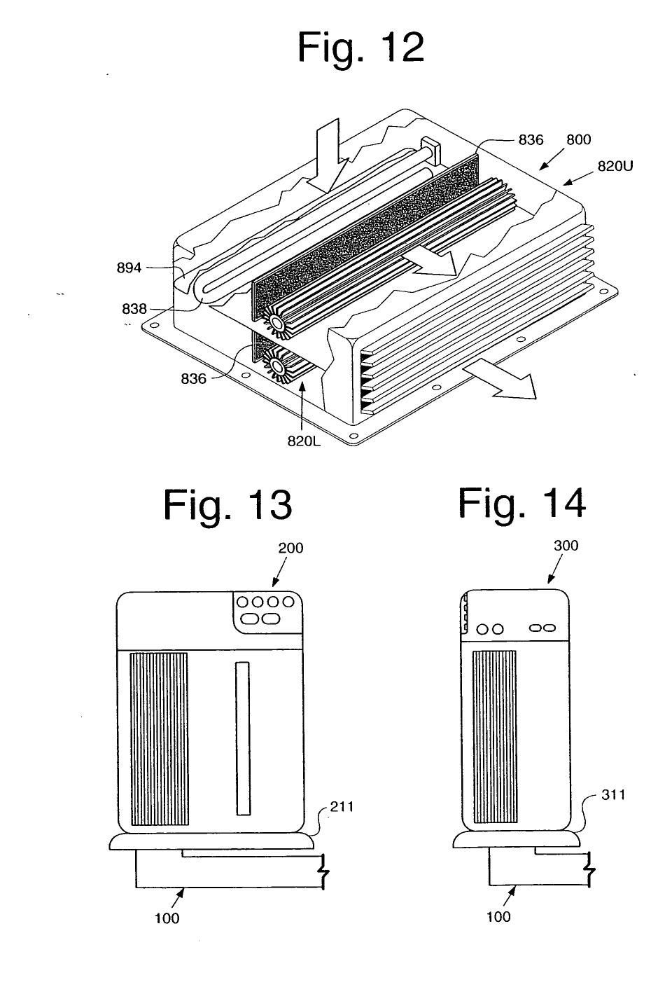

FIG. 12 illustrates an alternative embodiment of the air purification system 800. In the embodiment depicted in FIG. 11, a single U-shaped UV light 838 replaces the upper light 824U and the lower light 824L. The lamp may have a wide variety of shapes above and below the partition and still be a single lamp. An additional bend or two for example, may add intensity within the air treatment chambers. A U-shaped lamp 838 was chosen because of its availability and inherent simplicity. The U-shaped light 838 straddles the partitioning member 894 such that one leg is above the partitioning member 894 and the other is below it. The use of a single UV light would eliminate a component, and, therefore, save costs.

Tower

Referring now to FIGS. 13 and 14, it is contemplated that a space saving design, taller rather than shorter with controls easily accessible and at a comfortable distance from the floor but within reach, may be advantageous in order to make the product more appealing to consumers. FIG. 13 illustrates a first configuration of the “tower” model, and FIG. 14 illustrates a second configuration. In the embodiment depicted in FIG. 13, the air purification system 200 is aligned with the duct 100. However, the air purification system 300 is transverse to the duct 100 in the embodiment depicted FIG. 14. The air purifier system 200, 300 is tall and slender and has a base 211, 311 sized appropriately to cover the ventilation duct 100 in the room and also large enough to stabilize the air purifier system 200, 300. The base 211, 311 has several adjustable louver mechanisms (not shown) which can be manually and/or automatically adjusted via sensors, flow, temperature, timer, etc. working in conjunction with an operator programmed control to bring about a mechanically actuated means to control airflow and/or direct airflow.

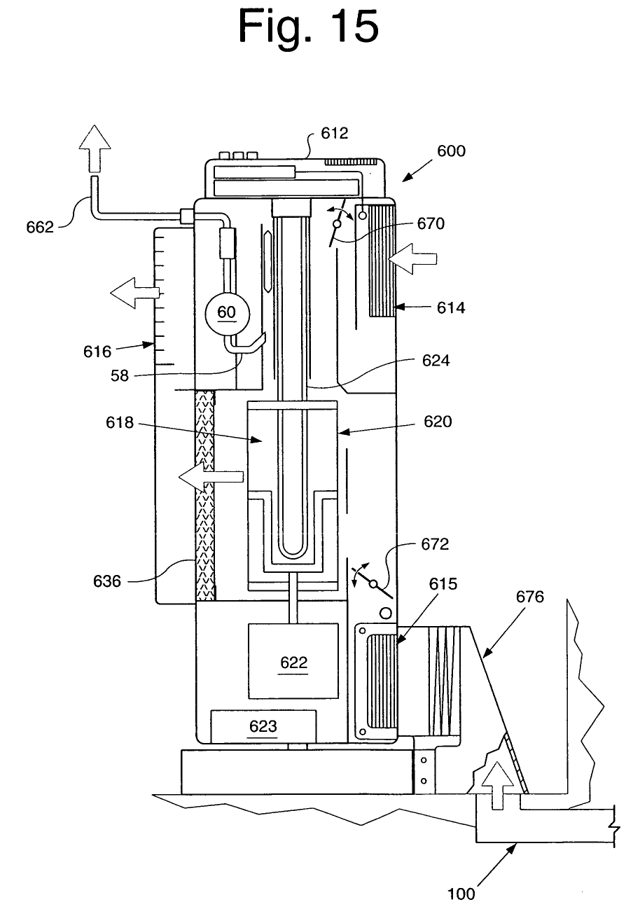

FIG. 15 illustrates an alternative “tower” version of the air purifier system, generally labeled with numeral 600. The air purifier system 600 includes a housing 612, a first inlet 614, a second inlet 615, and an outlet 616. As examples, the first inlet 614 may be connected to ambient air and the second inlet 615 may receive air from the ventilation duct 100. In this manner, the air purification system 600 may receive unpurified air from more than one source.

In some embodiments, the air purifier system 600 may include an adjustable duct 676. The adjustable duct 676 allows the user to connect the second inlet 615 to the ventilation duct 100 and place the air purification system 600 at a distance from the ventilation duct 100. Because users may have different needs and desires, the adjustable duct 676 is accordion-shaped such that it may expand or contract to achieve a desired spacing.

The air purifier system 600 includes a squirrel cage blower 620. In the depicted embodiment, the squirrel cage blower is generally vertical. A UV lamp 624 is mounted inside the squirrel cage blower 620. As such, the inside of the squirrel cage blower 620 forms the air treatment chamber 618. In some embodiments, the inside of the squirrel cage blower 620 may be comprised of a reflective material, such as polished aluminum, or coated with a UV inhibitor material. The reflective material intensifies the UV irradiation and promotes self-cleaning. In some embodiments, the air purifier system 600 includes a filter mesh 636. Additionally, the inside of the housing 612 may be reflective to assist in a self-cleaning manner by reflecting light on the outside of the squirrel cage blower 620. In lieu of a self cleaning feature, the device may have a washable ultraviolet light compatible (such as aluminum) germicidal irradiation sterile collection filter.

The air purification system 600 also includes a first damper 670 and a second damper 672. The first damper 670 is used to close-off the first inlet 614, and the second damper 672 is used to close-off the second inlet 615. As an example, the second damper 672 may be activated to prevent air from entering via the second inlet 615, thereby allowing only ambient air to enter through the first inlet 614.

In operation, the motor 622 rotates the squirrel cage blower 620 assisted by unit oscillator motor assembly 623. Unit oscillator motor assemblies are well-known. The squirrel cage blower 620 draws air into the housing 612 via the first inlet 614 and/or the second inlet 615. The air is treated by the UV lamp 624. Thereafter, the squirrel cage blower 620 pushes the air through the filter mesh 636 and out of the housing 612 via the outlet 616.

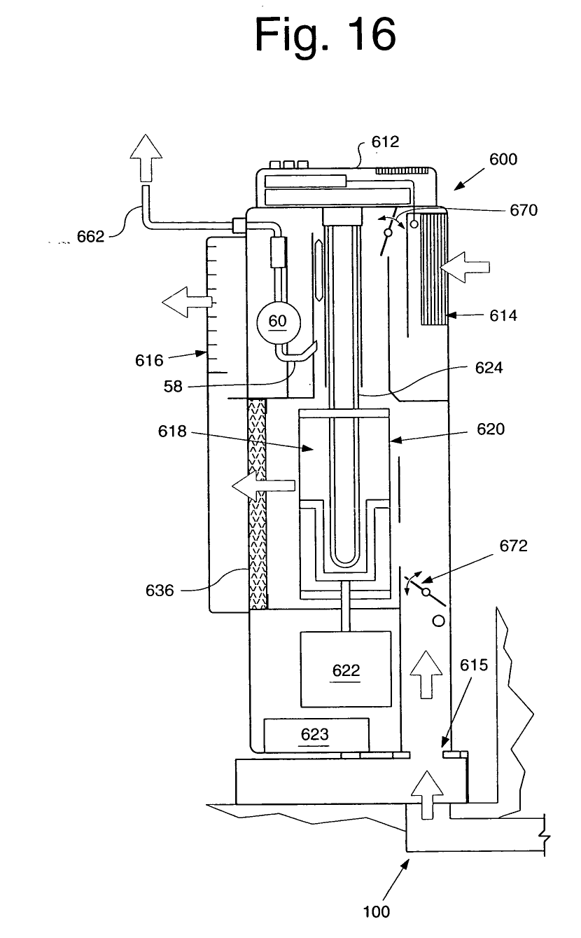

FIG. 16 illustrates an alternative embodiment of the air purifier system 600 wherein the second inlet 615 is directly connected to the ventilation duct 100.

Positive Pressure

The most efficient and maximum air purification is achieved within an enclosed room by creating a slightly positive pressure in the room. A positive pressure system reduces the overall air purification requirements of the room and provides for maximum air purification in the enclosed room. This may be extremely beneficial for organ transplant patients, people with allergies, and individuals who otherwise require an ultra-pure air environment.

Positive pressure prevents usual sources of indoor air contamination. Typically, air seeps into a room around windows, around electrical outlets, in the clearances under closed doors, and in the space between partially or fully opened doors. A positive pressure system prevents or reduces such air infiltration from minor leakage by reversing the airflow through these sources.

Positive pressure is achieved by bringing air into the room via a source of air from outside the room and forcing the room air to exit the room via a predetermined path, such as under a closed door, partially opened door, or pushed and drawn through an HVAC air return duct installed in the room. By limiting the amount air drawn from the room and using the ventilation duct 100 as a source of air outside of the enclosed room, it is possible to achieve a desired level of positive pressure within the room.

Partial or complete blockage of HVAC return air ducts may assist in overall efforts to achieve positive pressure. If the room is equipped with a return air duct and the duct is not flow-restricted, then the purified air within the room will follow the path of least resistance and exit the room through the return ventilation duct. However, if the return duct is blocked, then the purified air will tend to stay in the room or exit under a closed door.

The air purification system 10, 200, 300, 400, 500, 600, 800 greatly enhances the positive pressure system. By maintaining a positive pressure in the room and continuously circulating the air in the room through the air purification system 10, 200, 300, 400, 500, 600, unconditioned/unpurified air is prevented from rushing into the room while at the same time a high level of air quality within the room is maintained.

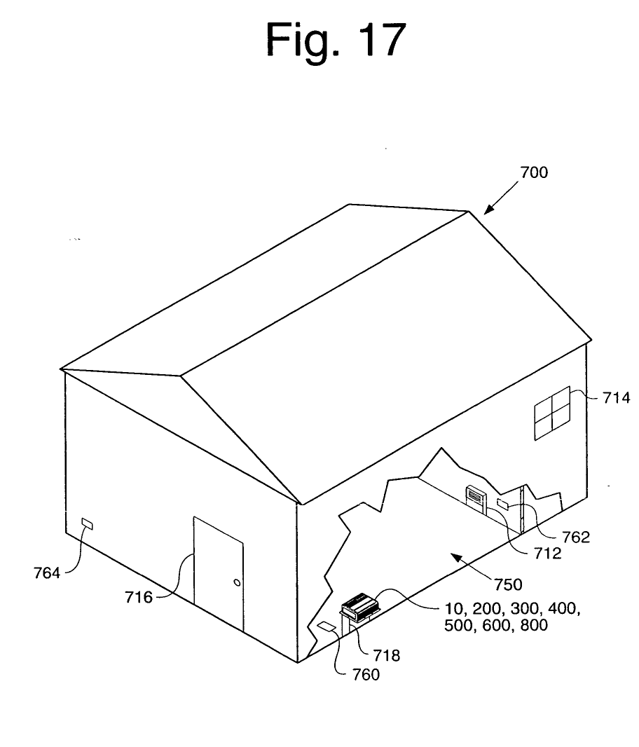

A positive pressure system is illustrated in FIG. 17. The positive pressure system 700 includes an enclosed space 750, a supply ventilation duct 718 connected to the enclosed space 750, and a return ventilation duct 712 connected to the enclosed space 750. Optionally, the enclosed space 750, such as a room, an office, a home, or a building, may have a door 716 or a window 714. The positive pressure system 700 also includes an air purification system 10, 200, 300, 400, 500, 600, 800 in fluid communication with the supply ventilation duct 718. Further, in the depicted embodiment, the air purification system 10, 200, 300, 400, 500, 600, 800 is in fluid communication with the ambient air present in the enclosed space 750.

In operation, the air purification system 10, 200, 300, 400, 500, 600, 800 draws air into the enclosed space 750 via the supply ventilation duct 718. The air purification system 10, 200, 300, 400, 500, 600, 800 simultaneously forces air out of the enclosed space 750. In general, air takes the path of least resistance and, so, air exits the enclosed space 750 via the return ventilation duct 712. However, in the depicted embodiment, the return ventilation duct 712 is either completely or partially blocked off. Thus, air is forced out of the enclosed space 750 via another path, such as under the door 716 or through gaps around the window 714. Those skilled in the art would understand that this would also be the situation if the enclosed space lacked the return ventilation duct 712. Because the volume of air entering the enclosed space 750 is greater than the air exiting the enclosed space, a positive air pressure within the enclose space 750 is achieved. Moreover, the air purification system 10, 200, 300, 400, 500, 600, 800 filters the ambient air in the enclosed space 750, thereby maintaining the quality of air within the enclosed space 750.

In the described positive pressure air purification system 700, it is also envisioned that some embodiments may include a measurement device 760 which compares the atmospheric pressure inside the enclosed space 750 versus the atmospheric pressure outside the enclosed space. In some embodiments, the measurement device 760 may be incorporated directly into the air purification system 10, 200, 300, 400, 500, 600, 800. A sensor device 762 in the enclosed space 750 and another remotely placed sensor 764 outside the enclosed space are connected to a microprocessor (not shown) of the measurement device 760. The sensors 762, 764 may be connected to the measurement device either by wire or wirelessly. It is envisioned that these sensors 762, 764 and the information they feed to the measurement device 760 can be used to control the air purification system 10, 200, 300, 400, 500, 600, 800. For example, the measurement device 760 may be programmed to alter the speed of the air handler or open the air inlet which is connected to the HVAC supply air duct upon sensing a certain pressure differential. This system will maximize the effectiveness of the air purification system and keep household pollutants out of the enclosed space as the device compensates for air infiltration. This also will allow the air purification system 10, 200, 300, 400, 500, 600, 800 to run at slower, more efficient and quieter speeds by monitoring and maintaining a desired pressure differential.

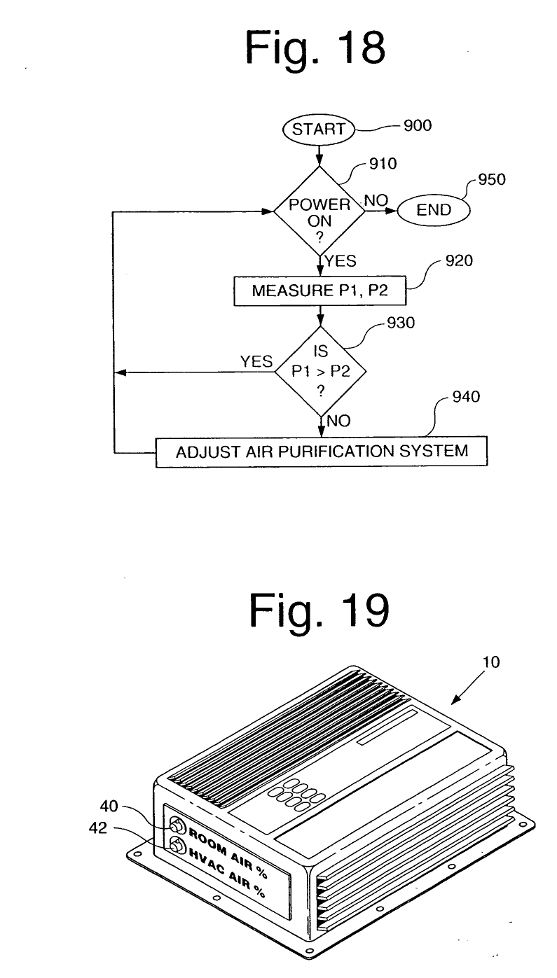

FIG. 18 illustrates a flow chart for the controller 90 of the measurement device 760. The measurement device 760 starts up in step 900 and proceeds to a first decision 910. In the decision 910, there is a decision whether the power is “on.” If the power is not “on” then the measurement device 760 stops in step 950. If the power of the air purification system 10, 200, 300, 400, 500, 600, 800 is “on,” then the measurement device 760 proceeds to the next step 920. In step 920, the measurement device 760 inquires the sensors 762, 764 to identify the current pressures inside the system and out. In step 930, there is a decision whether the pressure inside is greater than the pressure outside. If that is the case, then the measurement device 760 returns to step 910 and inquires whether the power of the air purification system is “on.” If, however, the pressure inside is equal to or less than the pressure outside, then in step 940 the measurement device 760 adjusts the air purification system 10, 200, 300, 400, 500, 600, 800 such that the pressure is increased within the system. Thereafter, the measurement device 760 returns to step 910. In this way the control system can maintain a positive pressure inside the system relative to outside.

Although ultraviolet air purification techniques are described for use in conjunction with the positive pressure system, other filtration means, woven filters, charcoal filters, electrostatic, electronic, charged collection plates, etc., are reasonable approaches to improving indoor air quality particularly when portable air purifiers employ source control measures exhaustively described.

Source Control

Referring now to FIGS. 19-25, the air purification system 10, 200, 300, 400, 500, 600, 800 may be adapted to receive air from more than one source. In such a case, the air purification system 10, 200, 300, 400, 500, 600, 800 may also include controls to select a source for purification.

The air purification system 10 can be placed over the ventilation duct 100 or some other source of indoor air pollution. As an example, in the embodiment depict in FIG. 1, the ventilation duct 100 is located on the floor and the second inlet 15 receives air from the floor mounted ventilation duct. However, the air purification system 10 could be placed in other locations. For example, the air purification system 10 may be mounted over an HVAC supply register located on a wall or ceiling. Typically, the air purifier system 10 sits several inches above the ventilation duct 100, and the air purifications system 10 may draw air completely or partially from the ventilation duct 100 and purify and filter the air prior to releasing the air into the room space.

When the air purification system 10 is placed over a ventilation duct 100, it draws a predetermined amount of air from the ventilation duct 100. In general, HVAC systems have their own filtration. Thus, the air purification system 10 is drawing in filtered air from the ventilation duct 100. However, the air purification system 10 could equally draw in unfiltered air from the ventilation duct 100. The intent of positioning the air purification system 10 to draw air from the ventilation duct 100 and purify/treat the air before it is released into the room is designed with the express intent to eliminate, to the extent possible, harmful contaminants that normally circulate into and through central HVAC systems. In fact, central HVAC systems can be a major source of indoor air quality concerns due to bio-nesting on filters and HVAC coils. Moreover, the HVAC system will draw unpurified air from other rooms via a supply duct and deliver the filtered but unpurified air to the air purification system 10, 200, 300, 400, 500, 600, 800 for purification. In this manner, the air purification system 10, 200, 300, 400, 500, 600, 800 takes advantage of the supply and return ducts of the HVAC system to address a major source of poor indoor air quality, namely the HVAC system itself. It is envisioned that additional ventilation ducts within a room may be blocked or attached via a connector to the air purification system 10. Further, additional ventilation ducts may be equipped with additional air purification systems.

Moreover, in one configuration, the air purifier system 10 may incorporate a louver with or without automated controls to simply direct air rising up from the floor duct into a horizontal flow redirected more or less, across the floor. As such, the air purifier system 10 may simply sit over a supply air duct and proceed in a “by-pass mode.”

Alternatively, the air purification system 10, 200, 300, 400, 500, 600, 800 may draw a predetermined amount of ambient air from the room. As an example, in the embodiment depicted in FIG. 2, the air purification system 10 draws ambient air in through the first inlet 14. If the room is enclosed, recirculating the ambient air through the air purification system 10, 200, 300, 400, 500, 600, 800 will greatly reduce the number of contaminants in the enclosed room.

Additionally, the air purification system 10, 200, 300, 400, 500, 600, 800 may draw air in from both the ventilation duct 100 and ambient air from the room. As such, the air purification system 10, 200, 300, 400, 500, 600, 800 may not only recirculate ambient air in a room, but also purify filtered air received from the ventilation duct 100.

Because the air purification system 10, 200, 300, 400, 500, 600, 800 may receive air from more than one source, the air purification may include a control mechanism to select one or more air sources for purification. The control mechanism may be automated or manual. Thus, in some embodiments, the air purification system 10 includes a mechanism to adjust the air inlet opening. By controlling the air inlet opening, it is possible to control the volume of air that is drawn in by the air handler, treated in the treatment chamber, and released back into the room.

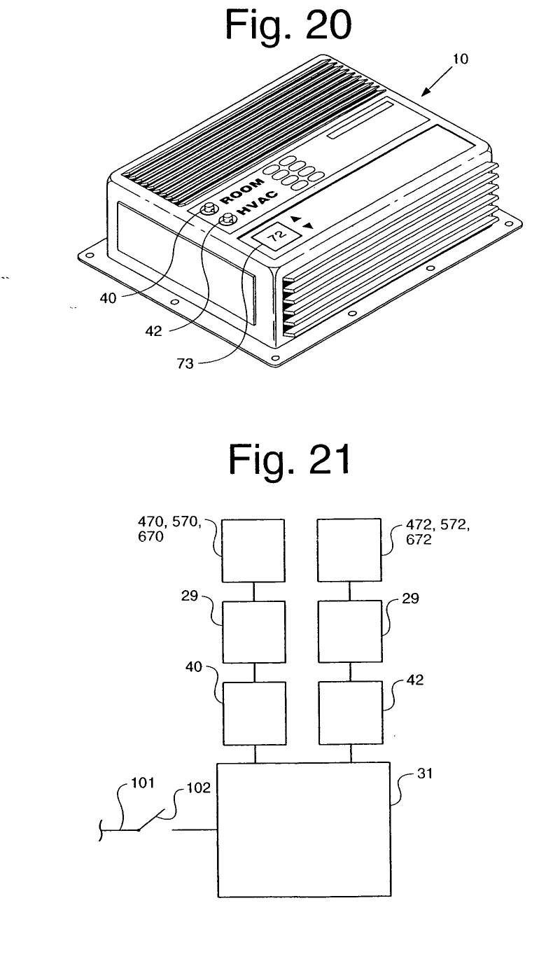

In the embodiment depicted in FIG. 19, the air purification system 10 includes a first switch 40 and a second switch 42. In the depicted embodiment, the first switch 40 is used to control the percentage of ambient air received through the first inlet, and the second switch 42 is used to control the amount of air received from the second inlet. While in the depicted embodiment the switches 40, 42 are placed on the side of the air purification system 10, the switches 40, 42 could also be placed on top of the air purification system 10 as shown in FIG. 20. Further, while only two switches are shown, those skilled in the art would understand that the air purification system may include a number of switches equal to the number of inlets. For example, if the air purification system had four inlets, it may also have four switches.

The switches 40, 42 may be used in several different ways. First, the switches 40, 42 may be used to control the dual air handler embodiment (best seen in FIGS. 9-12). In this configuration, the switch 40 is connected to the upper air handler 820U, and the switch 42 is connected to the lower air handler 820L. As such, the switches 40, 42 can control the respective speeds of the air handlers 820U, 820L. Obviously, the greater the speed of the air handler, the more air the air handler will draw in through an inlet. As such, a user may control the speed of an air handler to adjust the volume of air processed from a selected source.

Second, the switches 40, 42 may be connected to the louvered cover 17. For example, the louvers of the louvered cover 17 may be movable, and the switches 40, 42 may be used to control the amount of movement of the louvers. As an example, the switch 40 may be connected to an electro-mechanical actuator which is connected to the louvered cover 17. Rotation of the switch 40 would open or close the louvered cover 17, thereby opening or closing the inlet 14. Thus, a user could select an air source by rotating the switches 40, 42 to open or close the louvered covers 17.

Referring once again to FIG. 5, the switches 40, 42 are connected to the voltage regulator 31. Actuators 29 are electrically connected to the switches 40, 42 and the louvered cover 17 is mechanically connected to the actuators 29. As the switches 40, 42 are adjusted, the actuators 29 are displaced to open or close the louvered cover 17.

Third, the switches 40, 42 may be connected to dampers 470, 472, 570, 572, 670, 672. The dampers 470, 472, 570, 572, 670, 672 prevent air from entering the air treatment chamber from an air source, such as the first inlet or the second inlet. By controlling the dampers, it is possible to control the volume of air received from each air source. As an example, the first switch 40 may be connected electrically or mechanically to the first damper 670 of the air purification system 600 (as best seen in FIG. 16). Similarly, the second switch 42 may be connected to the second damper 672. A user may select to purifier air received by the first inlet 614, the second inlet 615, or some combination thereof. In the embodiment depicted in FIG. 16, the air purification system 600 includes the first damper 670 to control the percentage of ambient air received through the first inlet 614, and a second damper 672 to control to control the amount of air through the second inlet 615. By rotating the switches 40, 42, the user can adjust the opening of the respective damper 670, 672. The user could completely open the dampers 670, 672, completely close the dampers 670, 672, or partially open the dampers 670, 672. As such, the user may select the percentage of air received from each source.

FIG. 21 illustrates schematically the switches 40, 42 and the dampers 470, 472, 570, 572, 670, 672. The switches 40, 42 are electrically connected to the voltage regulator 31. Actuators 29 are electrically connected to the switches 40, 42. The dampers 470, 472, 570, 572, 670, 672 are mechanically connected to the actuators 29. As the switches 40, 42 are adjusted, the actuators 29 are displaced such that the dampers 470, 472, 570, 572, 670, 672 are open or closed.

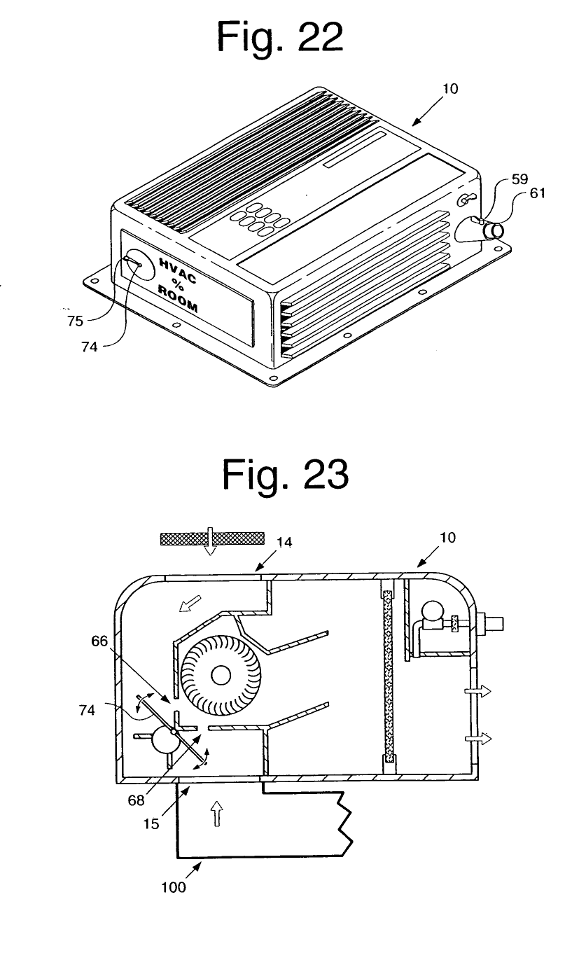

FIGS. 22 and 23 illustrate yet another apparatus for source control. In the embodiment depicted in FIGS. 22 and 23, the air purifier system 10 includes a first opening 66, a second opening 68, and a rotatable damper 74. The first opening 66 is in fluid communication with the first inlet 14, and the second opening 68 is in fluid communication with the second inlet 15. The rotatable damper 74 is rotatable such that it may be rotated to close the first opening 66 or the second opening 68. As best seen in FIG. 22, a user grasps a lever 75 to rotate the damper 74. The user may rotate the damper 74 in a first direction to select more air from the first air inlet 14, or the user may rotate the damper 74 in a second direction to select more air from the second inlet 15. The user may rotate the damper 74 to completely close the first opening 66 or the second opening 68. As such, the user may use the damper 74 to select a first air source, a second air source, or some combination thereof.

Temperature Control

It is also contemplated that for comfort reasons, the air purification system 10, 200, 300, 400, 500, 600, 800 may also contain common technologies used in room air heaters. For example, the air purification system 10, 200, 300, 400, 500, 600, 800 may incorporate a 750-1000 Watt ceramic heating element. This added feature is another step toward room air conditioning. Temperature settings may be user adjustable and may be automatically maintained based upon preset temperature requirements by the user. As an example, the embodiment depicted in FIG. 20 includes a thermostat 73 which the user may use to select a desired temperature.

The air purification system 10, 200, 300, 400, 500, 600, 800 may include a control system to achieve a variety of air purification and air handling requirements. As examples, the air purification system may have temperature sensors, thermostats, zone louvers, gear motors, positions sensors, switches, relays, variable speed motors, circuit boards, and microprocessors as part of its control system.

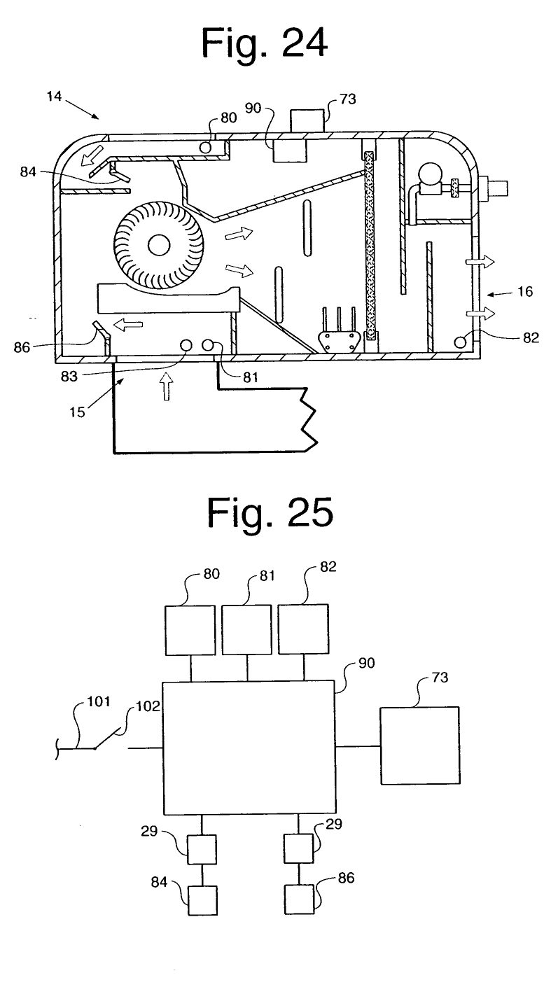

As best seen in FIG. 24, the air purification system 10, 200, 300, 400, 500, 600, 800 may incorporate a first inlet temperature sensor 80, a second inlet temperature sensor 81, and an outlet temperature sensor 82. Some embodiments may also include a flow sensor 83. As an example, the temperature sensor and the flow sensor may be obtained from Smarthome, Inc., 16542 Millikan Avenue, Irvine, Calif., U.S.A. The first inlet temperature sensor 80 is mounted adjacent the first inlet 14. The second inlet temperature sensor 81 is mounted adjacent the second inlet 15. The outlet temperature sensor 82 is mounted adjacent the outlet 16. The temperature sensors 80, 81, 82 are connected to the thermostat 73 (best seen in FIG. 20) and a controller 90. A first inlet damper 84 and a second inlet damper 86 are also connected to the controller 90. As an example, the dampers 84, 86 may controlled by pneumatic or electric actuators available from Greenheck Fan Corp., P.O. Box 410, Schofield, Wis., U.S.A. The first damper 84 is adjustable to restrict airflow from the first inlet 14. Similarly, the second damper 86 is adjustable to restrict airflow from the second inlet 15. A user adjusts the thermostat 73 to select a desired output temperature, and the controller 90 operates the first inlet damper 84 and the second inlet damper 86 to achieve the desired temperature output. Thus, the controller 90 will identify a desired outlet temperature based upon the thermostat 73 setting, identify existing temperature settings by receiving signals from the temperature sensors 80, 81, 82 and automatically adjust the first inlet damper 84 and the second inlet damper 86 until the desired outlet temperature is reached.

FIG. 25 illustrates schematically the controller 90. The controller 90 is electrically connected to a power source 101. In some embodiments, a switch 102 may be in-between the power source and the controller 90. The sensors 80, 81, 82 are electrically connected to the controller 90. The thermostat 73 is also electrically connected to the controller 90. Actuators 29 are electrically connected to the controller 90. The dampers 84, 86 are mechanically connected to the actuators 29. The controller 90 electrically powers the actuators 29 such that the actuators 29 are displaced and operate the dampers 84, 86.

In a first example, the air provided to the second inlet 15 may be considerably warmer than the room air. If the user desires a warmer output, the user adjusts the thermostat 73 such that more air is received from the second inlet 15. In other words, the user adjusts the thermostat 73, the controller 90 receives a signal from the thermostat 73, and the controller 90 accordingly adjusts the dampers 84, 86. However, if the user desires a cooler output, the user adjusts the thermostat 73 such that less air is received from the second inlet 15.

In a second example, the air provided to the second inlet 15 may be considerably cooler than the room air. If the user desires a cooler output, the user adjusts the thermostat 73 such that more air is received from the second inlet 15. In other words, the user adjusts the thermostat 73, the controller 90 receives a signal from the thermostat 73, and the controller 90 accordingly adjusts the dampers 84, 86. However, if the user desires a warmer output, the user adjusts the thermostat such that less air is received from the second inlet 15.

Compressor and Accompanying Attachments

It is also envisioned that the air purification system 10, 200, 300, 400, 500, 600, 800 may be equipped with a compressor 60 (best seen in FIG. 3). Tubing 62, such as small bore tubing or large bore tubing, is connected to the compressor 60. As examples, the compressor 60 may be a 120 Volts Alternating Current (VAC) 60 Hertz 1.8 Ampere compressor with an outlet pressure of about five pounds per square inch (psi) and a flow of 20 liters per minute. The small bore tubing may be approximately 3/16 of an inch in diameter, and the large bore tubing may be 13/16 of an inch in diameter. The compressor 60 and the tubing 62 can be used to route air from the air treatment chamber 18, 418, 518, 618, 818 directly to a personal device, such as a partial mask, a cannula, a full mask, a nebulizer, or other similar device. A high flow, low pressure blower used in CPAP-type machines is preferred to push air down a length of large bore 13/16 inch tubing to the inlet port of a CPAP-type machine.

In the depicted embodiments, the compressor 60 draws in treated air through a compressor inlet tube 58. In the embodiment depicted in FIG. 3, the compressor inlet tube 58 draws in air from behind the air guide 26 and adjacent the lamp 24. In a second embodiment depicted in FIG. 4, the compressor inlet tube 58 draws in air adjacent the outlet 416. In a third embodiment depicted in FIG. 5, the compressor inlet tube 58 draws in air next to the filter mesh 536 and above the air handler 520. In a fourth embodiment depicted in FIG. 7, the compressor inlet tube 58 draws in air next to the outlet 16 and adjacent the aluminum plates 28. In a fifth embodiment depicted in FIG. 8, the compressor inlet tube 58 draws in air next to the upper air handler 820U. In a sixth embodiment depicted in FIG. 14, the compressor inlet tube 58 draws in air next to the UV lamp 624.

After the air is drawn in through the compressor inlet tube 58, it is compressed by the compressor 60 and pushed out through the tubing 62. In some embodiments, a debris filter 49 is located intermediate the compressor 60 and the tubing 62. The compressor 60 may contribute some impurities to the air stream, and the debris filter 49 removes most of these contaminates. Various devices may be connected to the tubing 62 to receive the purified and pressurized air.

It is also envisioned that a smaller version of the air purification system 10, 200, 300, 400, 500, 600, 800 may be constructed. As shown in FIG. 31, smaller versions may be constructed using nebulizer features and components. The smaller air purification system would not attach to the ventilation duct 100, but instead would deliver adequately pressurized, purified air to the area of a user's mouth and nose. This unit may also be placed near, attached to, or installed inside and oxygen concentrator/generator. It may also attach to positive airway pressure machinery such as CPAP, Bilevel, ventilators, etc. and may or may not be required, based on proximity to these devices to deliver a pressurized source of purified air, i.e., these machines could draw air through our device for purification without the need for delivery under pressure. The smaller version may or may not incorporate ultraviolet germicidal irradiation air purification technology. Further, some versions may incorporate a fine weave bacteria filter to reduce a majority of pollens, some bacteria, house dust producing allergens, and other contaminants. Additional accessories may be incorporated into this device, including the accessories described below in conjunction with the description of FIG. 31.

It is noted that one oxygen concentrator has an external air port. It may have a filter also, but there is no UV component and does not have a flow controller or flow meter. The external air port has been available for at least 20 years. The oxygen concentrator could supply the air necessary, and the miniature units of the present invention could supply enhanced purification and a flow meter. Therefore, it is conceived of a new oxygen concentrator equipped with an internal UV air purification technology inside the unit, and a patient air flow meter on the exterior of the oxygen concentrator unit. This new oxygen concentrator would complement the oxygen/purified air delivery masks and cannulas of the present invention.

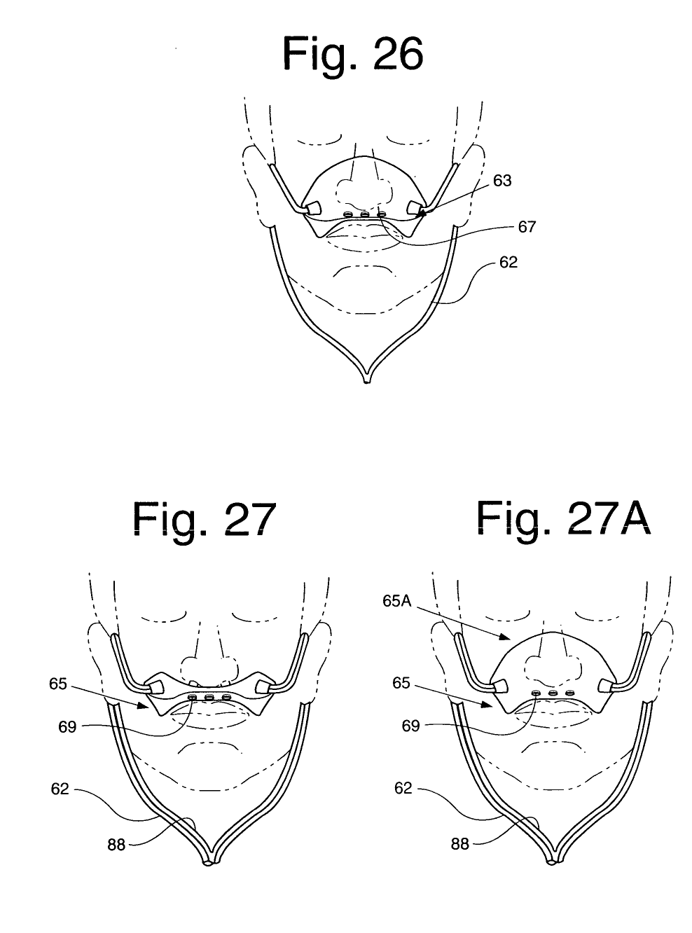

As best seen in FIG. 26, a nose mask 63 is connected to the tubing 62. Purified air is pushed through the tubing 62 by the compressor 60 to the nose mask 63. As such, a user may receive purified air directly. In some embodiments, the nose mask 63 includes air holes 67, which may provide some purified air to the mouth area of the user.

Referring now to FIGS. 27 and 27A, a cannula 65 is connected to the tubing 62. Purified air is pushed through the tubing 62 by the compressor 60 to the cannula 65. The cannula 65 fits under the nose of a user and directs pure air in the area around the nose for inhalation. The cannula 65 incorporates standard nasal cannula oxygen delivery prongs. As such, a user may receive purified air directly. As an example, the cannula may be a nasal cannula available from Hudson RCI, 27711 Diaz Road, P.O. Box 9020, Temecula, Calif., U.S.A. In some embodiments, the cannula 65 includes air holes 69, which may provide some purified air to the mouth area of the user. FIG. 27A incorporates the additional detachable mask feature as implemented by loop 65A.

Further, some embodiments may include a second tubing 88 connected to the cannula 65. The second tubing 88 may deliver a supplemental gas, such as oxygen, from a tank 79 (best seen in FIG. 23c) or any other therapeutic oxygen source to the cannula 65. Purified air delivery with supplemental oxygen will, in some cases, reduce the oxygen volume requirement while still maintaining appropriate oxygen saturation levels.

Further, the tubing 62 and the second tubing 88 may be incorporated into a single dual lumen extension tubing. The dual lumen extension tubing could be up to 50 feet in length, more or less, allowing patients to ambulate throughout their homes and into other unpurified air environments while maintaining a mask of purified air and prescribed oxygen delivery. The length of the dual lumen extension tubing may depend upon the tubing size and the operating pressure of the system.

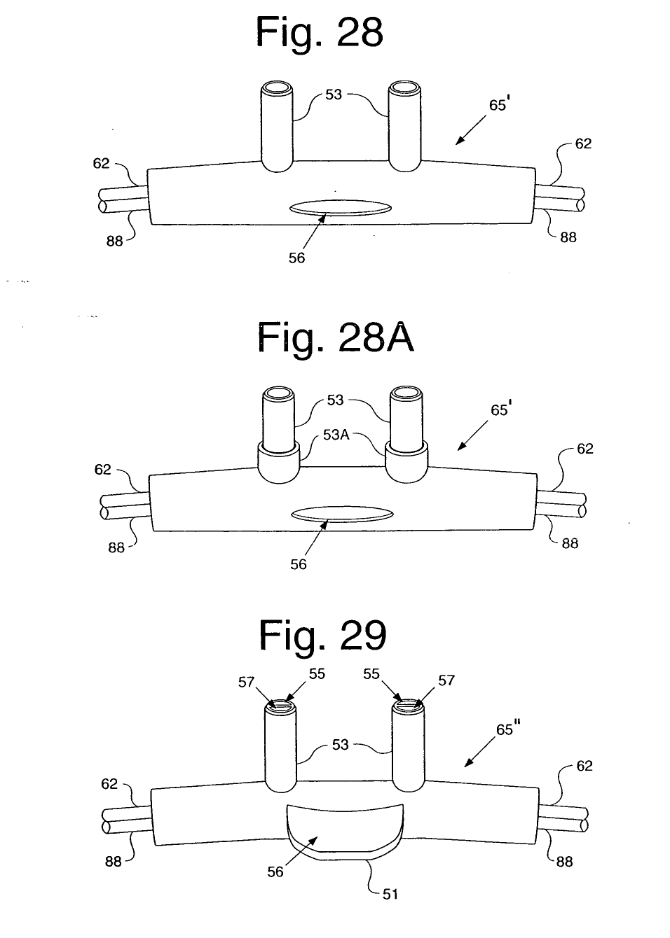

FIGS. 28, 28A, and 29 illustrate a modified cannula attached to both the tubing 62 and the second tubing 88. In the embodiment depicted in FIG. 28, a modified cannula 65′ is connected on each end to the tubing 62 and the second tubing 88. In the depicted embodiment, the tubing 62 and the second tubing 88 form a single tube. The cannula 65′ includes prongs 53 and an opening 56. In a first example, the second tubing 88 provides supplemental oxygen to the prongs 53 for inhalation through the nasal passages, whereas the tubing 62 delivers purified air to the opening 56 to provide purified air to the mouth area. However, with the exception of the embodiment of FIG. 28A, delivery may be reversed such that the second tubing 88 provides supplemental oxygen to the opening 56, and the tubing 62 delivers purified air to the prongs 53. In a third example, purified air from the tubing 62 mixes with the supplemental oxygen from the second tubing 88, and the prongs 53 and the opening 56 provide this mixture to the respective locations. FIG. 28A provides oxygen through 53 and purified air at and around 53A which is coincidentally the same air that is being supplied to opening 56. This configuration places oxygen first into the nares and then air into the nares. This configuration will not interfere with oxygen conservers which sense and deliver through 53 and while at the same time, a pure air cloud remains available-at the base of the nares via 53A and near the mouth via 56.

In the embodiment depicted in FIG. 29, a modified cannula 65″ is connected on each end to the tubing 62 and the second tubing 88. In the depicted embodiment, the tubing 62 and the second tubing 88 form a single tube. The cannula 65″ includes prongs 53 and an opening 56. However, each prong 53 is split such that it has a first port 55 and a second port 57. As an example, the second tubing 88 provides supplemental oxygen to the first port 55 for inhalation through the nasal passages, whereas the tubing 62 delivers purified air to the second port 57 for inhalation through the nasal passages. However, delivery may be reversed such that the second tubing 88 provides supplemental oxygen to the second port 57, and the tubing 62 delivers purified air to the first port 55. The cannula 65″ also includes the opening 56. Purified air, supplemental oxygen, or some combination thereof may be directed to the mouth area of a user via the opening 56. In the depicted embodiment, the cannula 65″ also includes a flap 51. The flap 51 is designed to direct the flow of air and/or oxygen towards the user's mouth area.

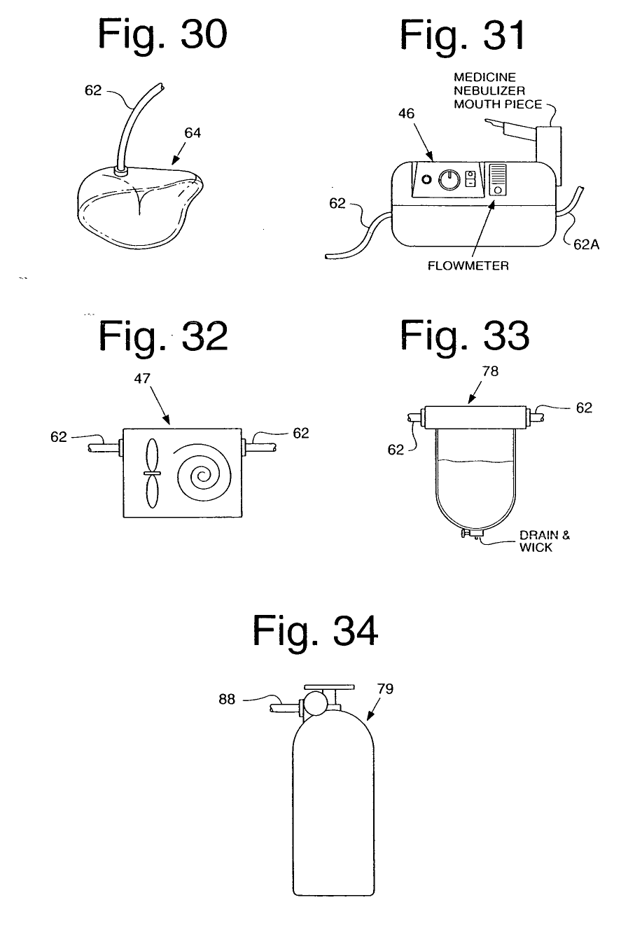

Referring now to FIG. 30, a face mask 64 is connected to the tubing 62. Purified air is pushed through the tubing 62 by the compressor 60 to the face mask 64. The face mask fits 64 fits over the nose and mouth of the user and provides purified air for inhalation. As such, a user may receive purified air directly. The mask 64 could also be worn on one's chin to direct a curtain over a person's mouth and into the nose. Conversely, a similar device could be applied to one's forehead to generate a blanket of air extending over the nose and mouth for inhalation directly from the air purification system 10, 200, 300, 400, 500, 600, 800.

FIG. 31 illustrates a nebulizer system consisting of a medication nebulizer 46 and the tubing 62. As an example, the nebulizer 46 may be a Salter 8900 Series medication nebulizer available from Salter Labs, 100 West Sycamore Road, Arvin, Calif., U.S.A. In general, a nebulizer is a device used to convert liquid medication to a fine mist that can be inhaled. When inhaled with purified air, the medication has a better chance to reach into the small airways of the lungs because the small airways are not reacting negatively to air contaminates. This increases the medication's effectiveness. Thus, a user may use the air purification system 10, 200, 300, 400, 500, 600, 800 for delivery of purified air to the nebulizer 46 (to the compressor air inlet 62A in FIG. 31) for treatment via inhalation.

Additional accessories may also be used with the nose mask 63, the face mask 64, the cannula 65, or within the nebulizer compressor 46. A purified air flow meter is included as shown in FIG. 31. Additional, a pure air inlet to the nebulizer compressor, 62A, is also added to supply purified air from the room air purifier of the present invention to a standard nebulizer compressor air inlet. For example, a cooling coil 47 (best seen in FIG. 32) may be placed intermediate the tubing 62. The cooling coil 47 allows ambient air to flow freely around the coil and cause the pressurized discharge air to return to an ambient air temperature for comfortable inhalation. As another example, a water trap 78 (best seen in FIG. 33) may be used to remove condensation from the tubing 62. The water trap 78 may be necessary when the purified air precipitates condensation upon cooling to ambient temperature. This may occur, as an example, when the air is highly pressurized due to the restrictiveness of significantly long, small bore tubing 62.

Referring once again to FIG. 24, some embodiments of the air purification system 10, 200, 300, 400, 500, 600, 800 may include a funnel 61. The funnel 61 may be connected to the compressor 60 or simply to the outlet of the air purification system. If equipped, this pressure flow intensifier may also have a valve 59 to regulate the flow of purified air at the user's face. In other words, a user would rotate the valve to increase or decrease the air flow from the funnel 61. The funnel 61 may be utilized to supply purified air to the inlet air supply opening of devices such as a Continuous Positive Airway Pressure (CPAP) system, a bilevel positive airway pressure (BiPAP) system, ventilators, nebulizers, or other personal, portable air protection/purification devices.

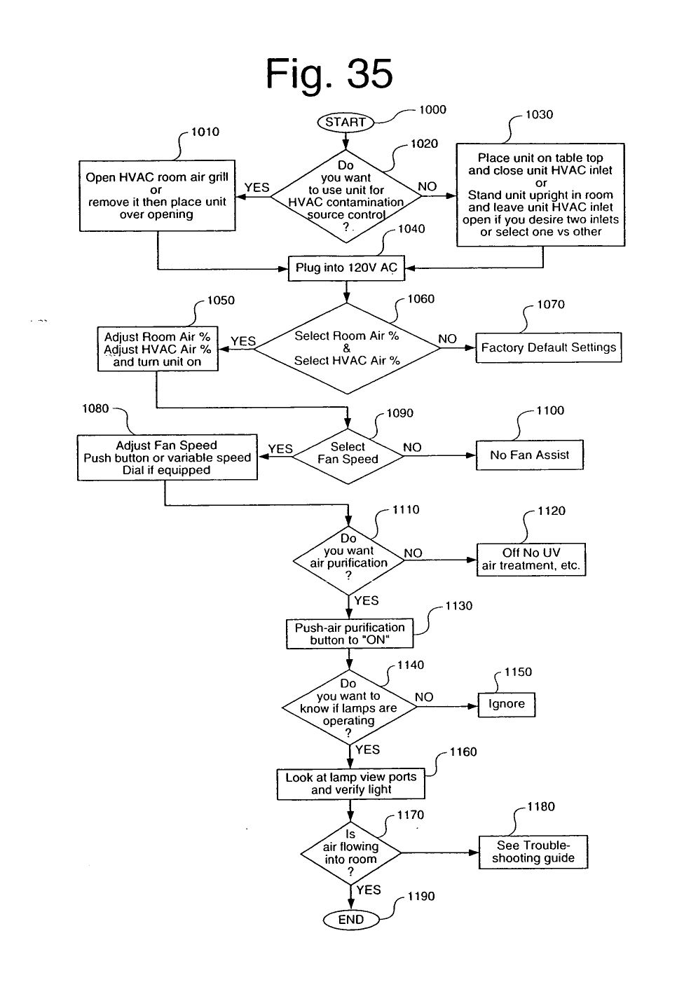

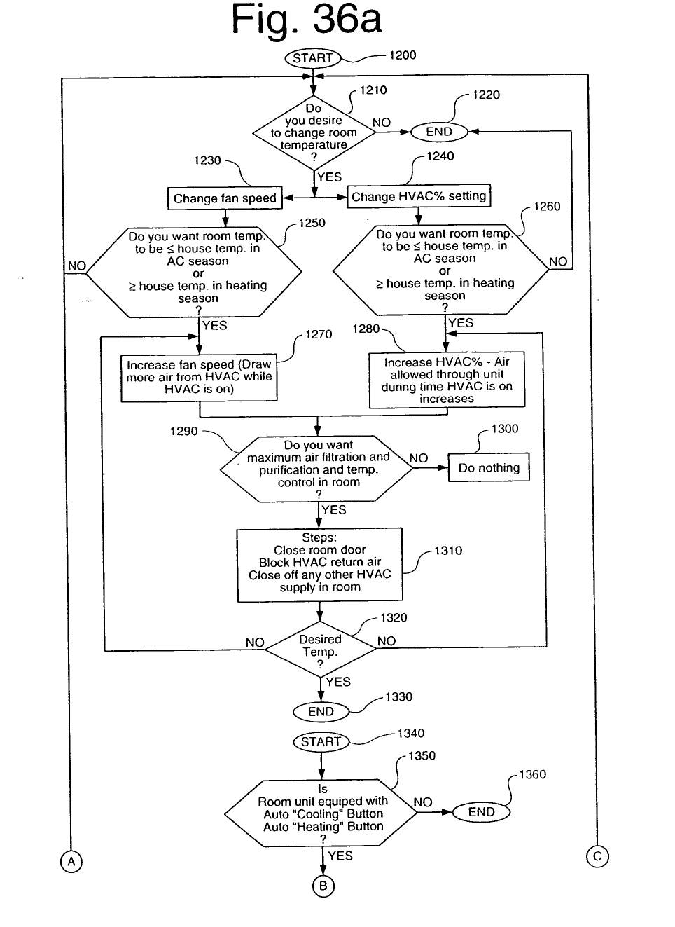

FIG. 35 illustrates a flowchart for operating the air purification system 10. In a first step 1000, the user begins the process of setting up the air purification system 10. In step 1020, the user decides if the user wants to use the air purification system 10 for HVAC contamination source control. If so, then the user will place the air purification system 10 over a ventilation duct in step 1010. Otherwise, the user will place the air purification system 10 on a table top or on the floor in step 1030. Thereafter, the user plugs in the air purification system 10 in step 1040.

step 1060, the user decides whether to adjust the air source selection settings. If not, the factory default settings will be selected in step 1070. Otherwise, the user will adjust the selected air source for air treatment in step 1050. As examples, the user may select 100% ambient air, 100% HVAC air, or some combination therebetween. After step 1060, the unit is turned on.

In step 1090, the user decides whether or not to adjust the fan speed. If not, then the factory setting remains in step 1100. Otherwise, the user will select a fan speed via a utton or switch in step 1080.

In step 1110, the user decides whether or not to engage the air purification option. If not, then the UV lamp is turned off is step 1120. Otherwise, the user pushes a button to turn the air purification on in step 1130.

In steps 1140, 1150, and 1160, the user may or may not verify that the ultraviolet lamp is operating.

In step 1170, the user decides whether the air purification system 10 is properly operating. If the system is not operating properly, the user consults a troubleshooting guide in step 1180. However, if the system is operating properly, the user continues to operate the air purification system 10 until it is powered off in step 1190.

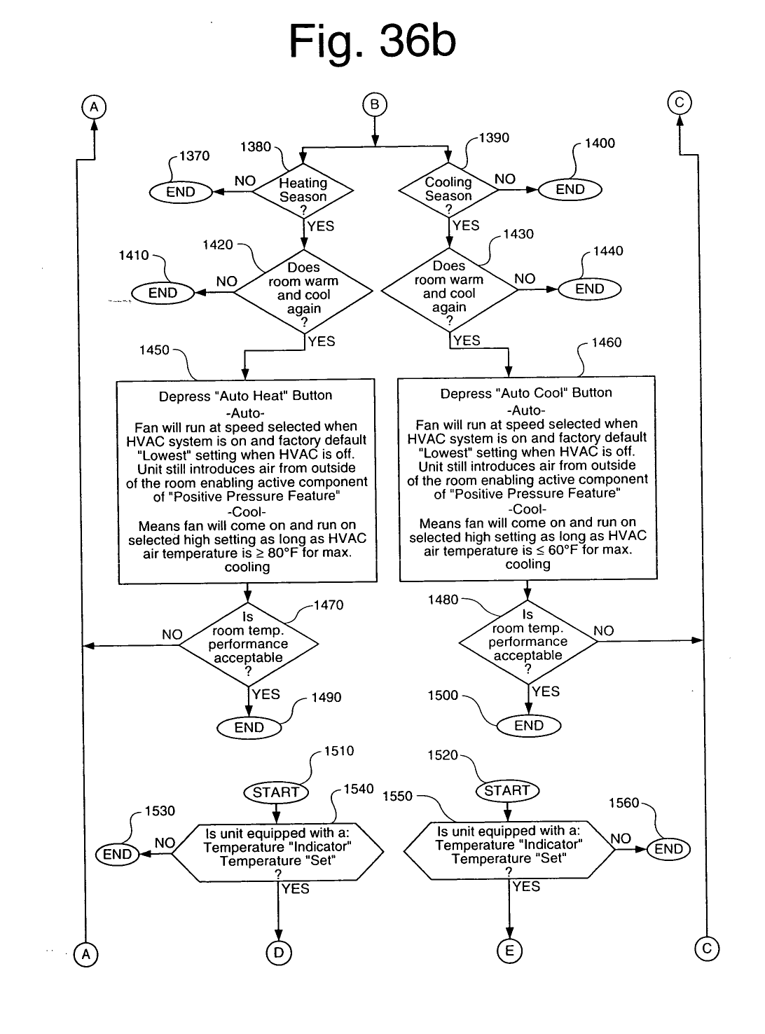

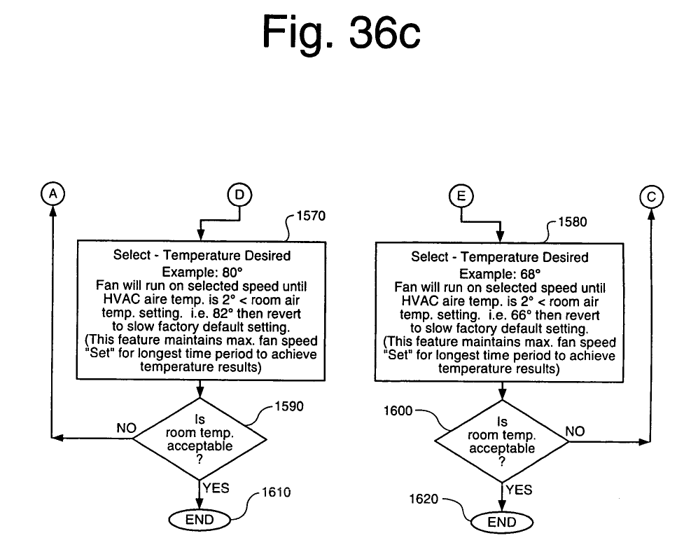

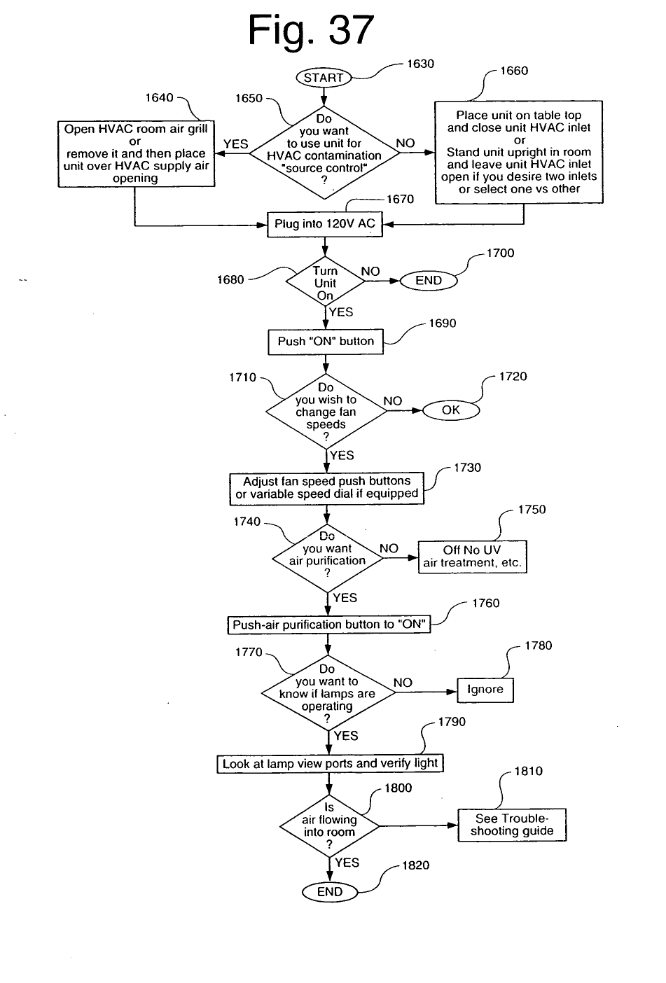

FIGS. 36a, 36b, and 36c illustrate a flowchart for maintaining a room temperature. In step 1200, the user begins by powering up the air purification system 10. In step 1210, the user decides whether or not to adjust the room temperature. If not, then the user stops in step 1220. However, if the user decides to change the room temperature, then a controller (not shown) changes the fan speed in step 1230 and changes the HVAC setting in step 1240. In steps 1250 and 1260, the controller decides if the room temperature should be less than or equal to the house temperature during summer months or greater than or equal to the house temperature in winter months. If not, then the controller returns to step 1210. If yes, then the controller increases the fan speed in step 1270. Additionally, the controller increases the HVAC percentage.

In step 1290, the user decides whether or not maximum air filtration is desired. If not, then no action is taken in step 1300. If yes, then the user must close the room door, block all room return air ducts, and close all other room HVAC ducts in step 1310.

The controller decides in step 1320 whether the desired room temperature has been obtained. If yes, then the process stops in step 1330. If not, then the controller returns to steps 1270 and 1280.

A process for automatically heating or cooling a room begins in step 1340. In step 1350, a user inquires whether the air purification system 10 is equipped with an “auto” heating or cooling button. If not, the process ends in step 1360. In steps 1380 and 1390, the controller decides whether it is the heating season or the cooling season. In steps 1420 and 1430, the user decides whether the room warms and cools cyclically. In step 1450, the user depresses the “auto heat” button. In step 1460, the user depresses the “auto cool” button. Thereafter, the user decides whether the room temperature is acceptable in steps 1470 and 1480. If not, then the user returns to step 1210. Otherwise the process ends in steps 1490 and 1500.

A process to select a desired temperature begins at steps 1510 and 1520. In steps 1540 and 1550, the user decides whether the air purification system 10 includes a mechanism to set the temperature. If not, then the process ends in steps 1530 and 1560. Otherwise, the user selects a desired temperature in steps 1570 and 1580. In steps 1590 and 1600, the user decides whether the room temperature is acceptable. If not, then the user returns to step 1210. Otherwise the process ends in steps 1610 and 1620.

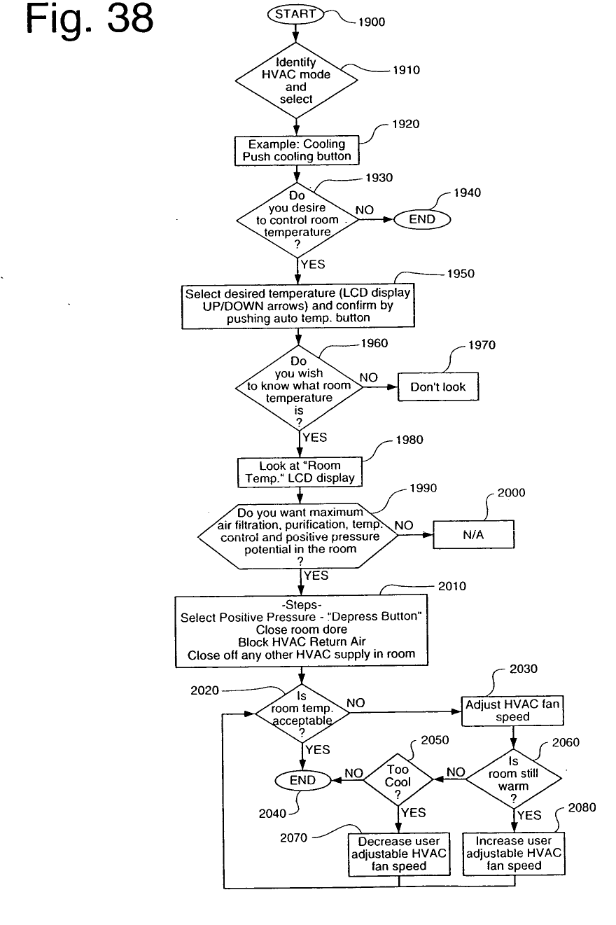

FIG. 37 illustrates a flowchart for setting up the air purification system 10. The process begins in step 1630. In step 1650, the user decides whether or not to use HVAC source contamination control. If not, then the user places the air purification system 10 on a table top or on the floor in step 1660. Otherwise, the user places the air purification system 10 over the HVAC supply duct in step 1640. In step 1670, the user plugs in the air purification system 10. In step 1680, the user decides whether or not to turn the air purification system 10 on. If not, then the process ends at step 1700. If so, then the user pushes the “on” button in step 1690.

Next, the user decides whether or not to change the fan speed in 1710. If not, then the user takes no action in step 1720. Otherwise, the user adjusts the fan speed in 1730. Then the user decides whether or not to turn on the air purification in step 1740. If not, then the controller turns off the ultraviolet lamp in step 1750. If so, then the user pushes the air purification button in step 1760. In step 1770, the user decides whether or not to verify operation of the lamp in step 1770. If not, no action is taken in step 1780. Otherwise, the user looks at the lamp view ports and verifies that there is a light in step 1790. In step 1800, the user decides whether the air purification system 10 is properly operating. If the system is not operating properly, the user consults a troubleshooting guide in step 1810. However, if the system is operating properly, the user continues to operate the air purification system 10 until it is powered off in step 1820.

FIG. 38 illustrates a flowchart for maintaining a room temperature utilizing the air purification system 10. The process begins at a step 1900. In step 1910, the user identifies the HVAC mode and selects an appropriate mode. For example, this may be a heating or cooling mode. In step 1920, the user pushes the appropriate button relating to the HVAC mode. In step 1930, the user decides whether or not to adjust the room temperature. If not, then the process ends in step 1940. Otherwise, the user adjusts the temperature in step 1950. In step 1960, the user decides whether or not he or she wishes to know the room temperature. In not, then the user does not look at the temperature in step 1970. In step 1980, the user looks at the temperature.

In step 1990, the user decides whether or not he or she wants maximum air filtration, purification, temperature control, and positive pressure. If not, no action is taken in step 2000. Otherwise, the user performs the following actions in step 2010: depress positive pressure button, close room door, block the HVAC return air duct, and close off any HVAC supply ducts. In step 2020, the user decides whether or not the room temperature is acceptable. If not, then the user adjusts the HVAC fan speed in step 2030. The user then decides whether or not the room is still warm in step 2060. If so, then the user increases the HVAC fan speed in step 2080. Otherwise, the user decides whether or not the room is too cool in step 2050. If so, then the user decreases the HVAC fan speed in step 2070. If the user decides that the room temperature is acceptable, then the process ends at step 2040.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated. The description of the invention is merely exemplary in nature and, thus, variations that do not depart from the gist of the invention are intended to be within the scope of the invention. Such variations are not to be regarded as a departure from the spirit and scope of the invention.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An air purification system for treating air, the system comprising:
a. a housing, said housing having a first inlet configured to receive ambient air, a second inlet adapted to receive air from a second source, and an outlet;
b. a treatment chamber mounted within said housing and in fluid communication with said first inlet and said second inlet;
c. at least one ultraviolet light disposed within said treatment chamber; and
d. at least one air handler in fluid communication with at least one of said first inlet and said second inlet, for moving air from one of said first inlet and said second inlet, through said treatment chamber and then through said outlet of said housing.

2. The air purification system of claim 1, further comprising a second ultraviolet light disposed on said treatment chamber.

3. The air purification system of claim 1, further comprising a second air handler in fluid communication with said treatment chamber.

4. The air purification system of claim 1, further comprising a removable inlet filter.

5. The air purification system of claim 1, further comprising a mesh filter in fluid communication with said treatment chamber.

6. The air purification system of claim 1, further comprising at least one louvered cover.

7. The air purification system of claim 1, further comprising at least one air guide located within said treatment chamber.

8. The air purification system of claim 1, further comprising:
a. an ultraviolet light baffle disposed between said air treatment chamber and said outlet; and
b. a power supply connected to said ultraviolet light baffle.

9. The air purification system of claim 1, wherein said air handler is selected from the group consisting of a tangential blower, a squirrel cage blower, or fan.

10. The air purification system of claim 1, further comprising at least one damper located adjacent said first inlet or said second inlet.

11. The air purification system of claim 1, wherein said at least one air handler is located within said treatment chamber.

12. The air purification system of claim 1, wherein said at least one air handler is located in between said treatment chamber and said outlet.

13. The air purification system of claim 1, wherein said housing includes a back wall, and said back wall is coated with a reflective material.

14. The air purification system of claim 1, further comprising at least one negative ion generator located adjacent said outlet.

15. The air purification system of claim 1, further comprising charged aluminum plates located adjacent said outlet.

16. The air purification system according to claim 1, further comprising an adjustable duct operatively connected to said second inlet and to said ventilation duct.

17. The air purification system according to claim 1, further comprising a first sensor mounted adjacent said first inlet, a second sensor mounted adjacent said second inlet, a third sensor mounted adjacent to said outlet, and a thermostat.

18. The air purification system of claim 1, further comprising a funnel in fluid communication with said treatment chamber.

19. The air purification system of claim 1, further comprising at least one duct operatively connected to said housing.

20. The air purification system of claim 19, wherein there is a first duct and a second duct, said first duct and said second duct providing a tapered passageway.

21. The air purification system of claim 1, further comprising a compressor, a compressor inlet tube connected to said compressor and to said treatment chamber, and tubing connected to said compressor.

22. The air purification system of claim 21, further comprising a face mask connected to said tubing.

23. The air purification system of claim 21, further comprising a nebulizer connected to said tubing.

24. The air purification system of claim 21, further comprising an accessory selected from the group consisting of a cooling coil, a water trap, and an oxygen tank.

25. The air purification system of claim 21, further comprising a nose mask connected to said tubing.

26. The air purification system of claim 25, wherein said nose mask includes a plurality of holes.

27. The air purification system of claim 26, further comprising a nasal cannula connected to said tubing.

28. The air purification system of claim 27, wherein said cannula includes a second tubing for the delivery of a supplemental gas.

29. The air purification system of claim 28, wherein said tubing is dual lumen extension tubing.

30. The air purification system of claim 29, further comprising a cannula connected to said dual lumen extension tubing.

31. The air purification system of claim 30, wherein said cannula includes two prongs and an opening.

32. The air purification system of claim 21, wherein each prong includes a first port and a second port.

33. The air purification system of claim 32, further comprising a flap.

34. An air purification system for purifying air, the system comprising:
a. a housing, said housing having a first inlet configured to receive ambient air, a second inlet connected to a ventilation duct, and an outlet;
b. a treatment chamber mounted within said housing and connected to said first inlet and said second inlet;
c. at least one ultraviolet light located within said treatment chamber;
d. an air handler for the movement of the air from said selected air source, through said treatment chamber, and out of said outlet;
e. a first damper in fluid communication with said first inlet;
f. a first actuator operatively connected to said first damper, said first actuator adapted to open and close said first damper;
g. a second damper in fluid communication with said second inlet;
h. a second actuator operatively connected to said second damper, said second actuator adapted to open and close said second damper; and
i. at least one switch electrically connected to said first damper and said second damper, wherein said at least one switch is used to activate at least one of said first actuator and said second actuator to select one or more sources for air purification.

35. An air purification system for treating air, the system comprising:
a. a housing, said housing having a first portion, a second portion, and a partition member located between said first portion and said second portion;
b. said first portion comprising:
i. a first inlet;
ii. a first air treatment chamber connected to said first inlet;
iii. a first outlet in fluid communication with said first air treatment chamber;
iv. a first air handler located between said first outlet and said first air treatment chamber;
c. said second portion comprising:
i. a second inlet;
ii. a second air treatment chamber connected to said second inlet;
iii. a second outlet in fluid communication with said second air treatment chamber;
iv. a second air handler located between said second outlet and said second air treatment chamber; and
d. at least one ultraviolet lamp at least partially located in said first air treatment chamber or said second air treatment chamber.

36. The air purification system according to claim 35, wherein there is a first ultraviolet lamp portion located in said first treatment chamber and a second ultraviolet lamp portion located in said second air treatment chamber, wherein said first and said second ultraviolet lamp portions may be part of separate or the same ultraviolet lamp.

37. The air purification system according to claim 35, wherein said at least one ultraviolet lamp is U-shaped, said U-shaped ultraviolet lamp having a first leg located within said first treatment chamber and a second leg located within said second treatment chamber.

38. The air purification system according to claim 35, wherein said partition member is ultraviolet light permeable.

39. An air purification system for treating air, the system comprising:
a. a housing, said housing having a first inlet configured to receive ambient air, a second inlet adapted to receive air from a second source, and an outlet;
b. a treatment chamber mounted within said housing and in fluid communication with said first inlet and said second inlet;
c. at least one U-shaped ultraviolet light disposed within said treatment chamber; and
d. at least one air handler in fluid communication with at least one of said first inlet and said second inlet, for moving air from one of said first inlet and said second inlet, through said treatment chamber and then through said outlet of said housing.

40. The air purification system according to claim 38, wherein said treatment chamber further comprises:
a. a first treatment chamber;
b. a second treatment chamber;
c. wherein one leg of said U-shaped ultraviolet light is disposed in said first treatment chamber, and another leg of said U-shaped ultraviolet light is disposed in said second treatment chamber.

41. An air purification system for treating air, the system comprising:
a. a housing, said housing having an upper portion, a lower portion, and a partition member located between said first portion and said second portion;
b. said first portion comprising:
i. a first inlet;
ii. a first air treatment chamber connected to said first inlet;
iii. a first outlet in fluid communication with said first air treatment chamber;
iv. a first air handler located between said first outlet and said first air treatment chamber;
c. said second portion comprising:
i. a second inlet;
ii. a second air treatment chamber connected to said second inlet;
iii. a second outlet in fluid communication with said second air treatment chamber;
iv. a second air handler located between said second outlet and said second air treatment chamber;
d. a first ultraviolet lamp portion at least partially located in said first air treatment chamber;
e. a second ultraviolet lamp portion at least partially located in said lower air treatment chamber; and
f. wherein said first and second ultraviolet lamp portions may be selectively and independently placed in an “on” or “off” position.

42. The system of claim 41, wherein said first and second ultraviolet lamp portions are selectively placed in an “on” or “off” position in order to extend the usable life of said lamp portions.

43. The system of claim 41, wherein said first and second ultraviolet lamp portions are selectively independently placed in an “on” and “off” position to emit light in any said chamber which is experiencing air flow.

44. The system of claim 41, further comprising:
a. An oxygen concentrator in fluid communication with one of said first inlet and said second inlet; and
b. an air source in fluid communication with the other of said first inlet and said second inlet.

45. An air purification system for treating air, the system comprising:
a. a housing, said housing having a first inlet configured to receive ambient air, a second inlet in fluid communication with an oxygen concentrator, and an outlet;
b. a treatment chamber mounted within said housing and in fluid communication with said first inlet and said second inlet;
c. at least one ultraviolet light disposed within said treatment chamber; and
d. at least one air handler in fluid communication with at least one of said first inlet and said second inlet, for moving air from one of said first inlet and said second inlet, through said treatment chamber and then through said outlet of said housing,
